(19) United States
(12) Patent Application Publication
(10) Pub. No.: US 2010/0173939 A1
(43) Pub. Date: Jul. 8, 2010

(54) ANTIVIRAL COMPOUNDS

(75) Inventor: John O. Link, San Francisco, CA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/639,404

(22) Filed: Dec. 16, 2009

Related U.S. Application Data
(60) Provisional application No. 61/140,030, filed on Dec. 22, 2008.

(51) Int. Cl. A61K 31/4709; C07D 245/04; A61P 31/12

(52) U.S. Cl. 514/312; 540/460

(57) ABSTRACT

The present invention relates to macrocyclic compounds of formula (I) that are useful as inhibitors of hepatitis C virus (HCV) NS3 protease, their synthesis, and their use for treating or preventing HCV infection.

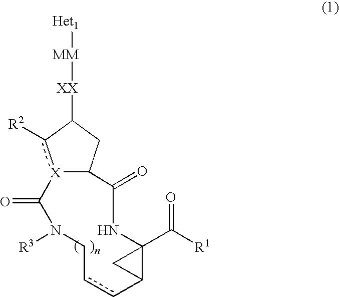

R¹ is:

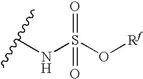

This application is filed under 35 U.S.C. 111(a) claiming the benefit under 35 U.S.C. 119(e) of U.S. provisional application 61/140,030 filed Dec. 22, 2008 of which is herein incorporated by reference in its entirety for all purposes.

The present invention is concerned with macrocyclic compounds having inhibitory activity on the replication of the hepatitis C virus (HCV). It further concerns compositions comprising these compounds as active ingredients as well as processes for preparing these compounds and compositions.

Hepatitis C virus is the leading cause of chronic liver disease worldwide and has become a focus of considerable medical research. HCV is a member of the Flaviviridae family of viruses in the hepacivirus genus, and is closely related to the flavivirus genus, which includes a number of viruses implicated in human disease, such as dengue virus and yellow fever virus, and to the animal pestivirus family, which includes bovine viral diarrhea virus (BVDV). HCV is a positive-sense, single-stranded RNA virus, with a genome of around 9,600 bases. The genome comprises both 5′ and 3′ untranslated regions which adopt RNA secondary structures, and a central open reading frame that encodes a single polyprotein of around 3,010-3,030 amino acids. The polyprotein encodes ten gene products which are generated from the precursor polyprotein by an orchestrated series of co- and posttranslational endoproteolytic cleavages mediated by both host and viral proteases. The viral structural proteins include the core nucleocapsid protein, and two envelope glycoproteins E1 and E2. The non-structural (NS) proteins encode some essential viral enzymatic functions (helicase, polymerase, protease), as well as proteins of unknown function. Replication of the viral genome is mediated by an RNA-dependent RNA polymerase, encoded by nonstructural protein 5b (NS5B). In addition to the polymerase, the viral helicase and protease functions, both encoded in the bifunctional NS3 protein, have been shown to be essential for replication of HCV RNA. In addition to the NS3 serine protease, HCV also encodes a metalloproteinase in the NS2 region.

Following the initial acute infection, a majority of infected individuals develop chronic hepatitis because HCV replicates preferentially in hepatocytes but is not directly cytopathic. In particular, the lack of a vigorous T-Lymphocyte response and the high propensity of the virus to mutate appear to promote a high rate of chronic infection. Chronic hepatitis can progress to liver fibrosis leading to cirrhosis, end-stage liver disease, and HCC (hepatocellular carcinoma), making it the leading cause of liver transplantations.

There are 6 major genotypes and more than 50 subtypes, which are differently distributed geographically. HCV type I is the predominant genotype in Europe and the US. The extensive genetic heterogeneity of HCV has important diagnostic and clinical implications, perhaps explaining difficulties in vaccine development and the lack of response to therapy.

Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades.

Current HCV therapies are based on (pegylated) interferon-alpha (IFN-a) in combination with ribavirin. This combination therapy yields a sustained virologic response in more than, 40% of patients infected by genotype 1 viruses and about 80% of those infected by genotypes 2 and 3. Beside the limited efficacy on HCV type 1, this combination therapy has significant side effects and is poorly tolerated in many patients. Major side effects include influenza-like symptoms, hematologic abnormalities, and neuropsychiatric symptoms. Hence there is a need for more effective, convenient and better tolerated treatments.

Recently, two peptidomimetic HCV protease inhibitors have gained attention as clinical candidates, namely BILN-2061 disclosed in WO00/59929 and VX-950 disclosed in WO03/87092. A number of similar HCV protease inhibitors have also been disclosed in the academic and patent literature. It has already become apparent that the sustained administration of BILN-2061 or VX-950 selects HCV mutants which are resistant to the respective drug, so called drug escape mutants. These drug escape mutants have characteristic mutations in the HCV protease genome, notably D168V, D168A and/or A1565. Accordingly, additional drugs with different resistance patterns are required to provide failing patients with treatment options, and combination therapy with multiple drugs is likely to be the norm in the future, even for first line treatment.

Experience with HIV drugs, and HIV protease inhibitors in particular, has further emphasized that sub-optimal pharmacokinetics and complex dosage regimes quickly result in inadvertent compliance failures. This in turn means that the 24 hour trough concentration (minimum plasma concentration) for the respective drugs in an HIV regime frequently falls below the IC₉₀ or ED₉₀ threshold for large parts of the day. It is considered that a 24 hour trough level of at least the IC₅₀, and more realistically, the IC % or ED₉₀, is essential to slow down the development of drug escape mutants.

Achieving the necessary pharmacokinetics and drug metabolism to allow such trough levels provides a stringent challenge to drug design. The strong peptidomimetic nature of prior art HCV protease inhibitors, with multiple peptide bonds poses pharmacokinetic hurdles to effective dosage regimes.

There is a need for HCV inhibitors which may overcome the disadvantages of current HCV therapy such as side effects, limited efficacy, the emerging of resistance, and compliance failures.

The present invention concerns HCV inhibitors which are superior in one or more of the following pharmacological related properties, i.e. potency, decreased cytotoxicity, improved pharmacokinetics, improved resistance profile, acceptable dosage and pill burden.

In addition, the compounds of the present invention have relatively low molecular weight and are easy to synthesize, starting from starting materials that are commercially available or readily available through art-known synthesis procedures.

WO05/010029 discloses aza-peptide macrocyclic Hepatitis C serine protease inhibitors, pharmaceutical compositions comprising the aforementioned compounds for administration to a subject suffering from HCV infection, and methods of treating an HCV infection in a subject by administering a pharmaceutical composition comprising the said compounds.

The present invention concerns inhibitors of HCV replication, which can be represented by formula (I):

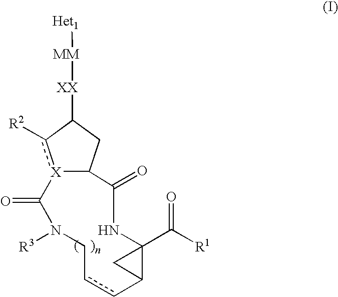

R¹ is:

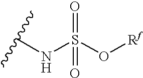

and the N-oxides, salts, and stereoisomers thereof, wherein each dashed line (represented by) represents an optional double bond;

X is N, CH and where X bears a double bond it is C;

R¹ is —NH—SO₂(OR¹);

R² is hydrogen, and where X is C or CH, R² may also be C₁₋₆alkyl;

R³ is hydrogen, C₁₋₆alkyl, C₁₋₆alkoxyC₁₋₆alkyl, C₃₋₇cycloalkyl;

R⁴ is aryl or Het;

n is 3, 4, 5, or 6;

carbon atoms bearing four substituents and including at least one bond to hydrogen in a compound of structure (I) may optionally have one or more of their hydrogen atoms replaced by halo where the halo can be F, Cl, Br or I, preferably F;

R⁵ represents halo, C₁₋₆alkyl, hydroxy, C₁₋₆alkoxy, polyhaloC₁₋₆alkyl, phenyl, or Het;

R⁶ represents C₁₋₆alkoxy, mono- or di-C₁₋₆alkylamino;

R⁷ is hydrogen; aryl; Het; C₃₋₇cycloalkyl optionally substituted with C₁₋₆alkyl; or C₁₋₆alkyl optionally substituted with C₃₋₇cycloalkyl, aryl or with Het;

R⁸ is aryl; Het; C₃₋₇cycloalkyl optionally substituted with C₁₋₆alkyl; or C₁₋₆alkyl optionally substituted with C₃₋₇cycloalkyl, aryl or with. Het;

aryl as a group or part of a group is phenyl or naphthyl optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or di-C₁₋₆alkylamino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkylpiperazinyl, 4-C₁₋₆alkylcarbonylpiperazinyl, and morpholinyl; wherein the morpholinyl and piperidinyl groups may be optionally substituted with one or with two C₁₋₆alkyl radicals;

Het as a group or part of a group is a 5 or 6 membered saturated, partially unsaturated or completely unsaturated heterocyclic ring containing 1 to 4 heteroatoms each independently selected from nitrogen, oxygen and sulfur, said heterocyclic ring being optionally condensed with a benzene ring; and Het as a whole being optionally substituted with one, two or three substituents each independently selected from the group consisting of halo, hydroxy, nitro, cyano, carboxyl, C₁₋₆alkyl, C₁₋₆alkoxy, C₁₋₆ alkoxyC₁₋₆alkyl, C₁₋₆alkylcarbonyl, amino, mono- or di-C₁₋₆alkylamino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkylpiperazinyl, 4-C₁₋₆alkylcarbonylpiperazinyl, and morpholinyl; wherein the morpholinyl and piperidinyl groups may be optionally substituted with one or with two C₁₋₆alkyl radicals;

R^{f} is A³,

Het¹ is a heterocycle or aryl group and can optionally be substituted with up to two Het and up to five groups selected independently from R⁴, R⁵ or R⁶;

MM is CO or a bond;

XX is O, NH, N(C₁₋₄) alkyl), a bond, or CH₂;

A³ is independently selected from PRT, H, —OH, —C(O)OH, cyano, alkyl, alkenyl, alkynyl, amino, amido, imido, imino, halogen, CF₃, CH₂CF₃, cycloalkyl, nitro, aryl, aralkyl, alkoxy, aryloxy, heterocycle, —C(A²)₃, —C(A²)₂—C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(OA²), —OCH₂P(Y¹)(A²)(OA²), —OCH₂P(Y¹)(A²)(N (A²)₂), —C(O)OCH₂P(Y¹)(OA²) (OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(Y¹)(OA²)(N(A²)₂), —CH₂P(Y¹)(N(A²)₂)(N(A²)₂), —C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₘ—O—C(O)—Oalkyl, —O—(CH₂), —O—C(O)—(CH₂)ₘ-alkyl, C(O)—O-alkyl, —(CH₂)ₘO—C(O)—O-cycloalkyl, —N(H)C(Me)C(O)O-alkyl, SRᵣ, S(O)Rᵣ, S(O)₂Rᵣ, or alkoxy arylsulfonamide, wherein each A³ may be optionally substituted with 1 to 4

—R¹¹¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A²)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘOC(O)Oalkyl, —O—(CH₂)ₘ—O—C(O)—(CH₂)ₘ-alkyl, —(CH₂)ₘ—O—C(O)—O-alkyl, —(CH₂)ₘ—O—C(O)-β-cycloalkyl, —N(H)C(CH₃)C(O)O-alkyl, or alkoxyarylsulfonamide, optionally substituted with R¹¹¹;

A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;

R¹¹¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;

Y¹ is independently O, S, N(A³), N(O)(A³), N(OA³), N(O)(OA³) or N(N(A³)(A³));

m is 0 to 6;

r is 0 to 6.

Compounds of the present invention can also be represented by formula (II):

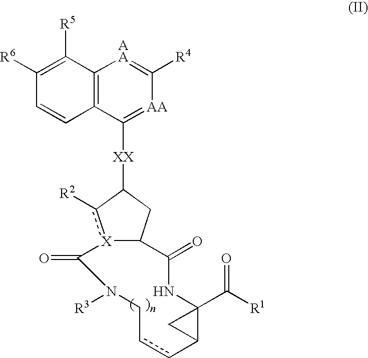

wherein AA is independently N or CH.

The invention further relates to methods for the preparation of the compounds of formula (I), the N-oxides, addition salts, quaternary amines, metal complexes, and stereochemically isomeric forms thereof, their intermediates, and the use of the intermediates in the preparation of the compounds of formula (I).

The invention relates to the compounds of formula (I) per se, the N-oxides, addition salts, quaternary amines, metal complexes, and stereochemically isomeric forms thereof, for use as a medicament. The invention further relates to pharmaceutical compositions comprising a carrier and an anti-virally effective amount of a compound of formula (I) as specified herein. The pharmaceutical compositions may comprise combinations of the aforementioned compounds with other anti-HCV agents. The invention further relates to the aforementioned pharmaceutical compositions for administration to a subject suffering from HCV infection.

The invention also relates to the use of a compound of formula (I), or a N-oxide, addition salt, quaternary amine, metal complex, or stereochemically isomeric forms thereof, for the manufacture of a medicament for inhibiting HCV replication. Or the invention relates to a method of inhibiting HCV replication in a warm-blooded animal, said method comprising the administration of an effective amount of a compound of formula (I), or a prodrug, N-oxide, addition salt, quaternary amine, metal complex, or stereochemically isomeric forms thereof.

The compounds of the invention have inhibitory activity toward HCV protease. Unexpectedly, it has been found that compounds possessing the acyl sulfamate group of the following formula:

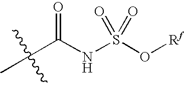

are suitably stable under physiological conditions. Additionally, it has been determined that representative compounds possessing this sulfamate group are unexpectedly potent inhibitors of HCV NS3 protease.

As used in the foregoing and hereinafter, the following definitions apply unless otherwise noted.

The term halo is generic to fluoro, chloro, bromo and iodo.

The term “polyhaloC₁₋₆alkyl” as a group or part of a group, e.g. in polyhaloC₁₋₆alkoxy, is defined as mono- or polyhalo substituted C₁₋₆alkyl, in particular C₁₋₆alkyl substituted with up to one, two, three, four, five, six, or more halo atoms, such as methyl or ethyl with one or more fluoro atoms, for example, difluoromethyl, trifluoromethyl, trifluoroethyl. Preferred is trifluoromethyl. Also included are perfluoroC₁₋₆alkyl groups, which are C₁₋₆alkyl groups wherein all hydrogen atoms are replaced by fluoro atoms, e.g. pentafluoroethyl. In case more than one halogen atom is attached to an alkyl group within the definition of polyhaloC₁₋₆alkyl, the halogen atoms may be the same or different.

As used herein “C₁₋₄alkyl” as a group or part of a group defines straight or branched chain saturated hydrocarbon radicals having from 1 to 4 carbon atoms such as for example methyl, ethyl, 1-propyl, 2-propyl, 1-butyl, 2-butyl, 2-methyl-1-propyl; “C₁₋₆alkyl” encompasses C₁₋₄alkyl radicals and the higher homologues thereof having 5 or 6 carbon atoms such as, for example, 1-pentyl, 2-pentyl, 3-pentyl, 1-hexyl, 2-hexyl, 2-methyl-1-butyl, 2-methyl-1-pentyl, 2-ethyl-1-butyl, 3-methyl-2-pentyl, and the like. Of interest amongst C₁₋₆alkyl is C₁₋₄ alkyl.

The term, “C₂₋₆alkenyl” as a group or part of a group defines straight and branched chained hydrocarbon radicals having saturated carbon-carbon bonds and at least one double bond, and having from 2 to 6 carbon atoms, such as, for example, ethenyl (or vinyl), 1-propenyl, 2-propenyl (or allyl), 1-butenyl, 2-butenyl, 3-butenyl, 2-methyl2-propenyl, 2-pentenyl, 3-pentenyl, 2-hexenyl, 3-hexenyl, 4-hexenyl, 2-methyl-2-butenyl, 2-methyl-2-pentenyl and the like. Of interest amongst C₂₋₆alkenyl is C₂₋₄alkenyl.

The term “C₂₋₆alkynyl” as a group or part of a group defines straight and branched chained hydrocarbon radicals having saturated carbon-carbon bonds and at least one triple bond, and having from 2 to 6 carbon atoms, such as, for example, ethynyl, 1-propynyl, 2-propynyl, 2-butynyl, 3-butyryl, 2-pentynyl, 3-pentynyl, 2-hexynyl, 3-hexynyl and the like. Of interest amongst C₂₋₆alkynyl is C₂₋₄alkynyl. C₃₋₇cycloalkyl is generic to cyclopropyl, cyclobutyl, cyclopentyl, cyclohexyl and cycloheptyl.

C₁₋₆alkanediyl defines bivalent straight and branched chain saturated hydrocarbon radicals having from 1 to 6 carbon atoms such as, for example, methylene, ethylene, 1,3-propanediyl, 1,4-butanediyl, 1,2-propanediyl, 2,3-butanediyl, 1,5-pentanediyl, 1,6-hexanediyl and the like. Of interest amongst C₁₋₆alkanediyl is C₁alkanediyl. C₁₋₆alkoxy means C₁₋₆alkyloxy wherein C₁₋₆alkyl is as defined above.

As used herein before, the term (=0) or oxo forms a carbonyl moiety when attached to a carbon atom, a sulfoxide moiety when attached to a sulfur atom and a sulfonyl moiety when two of said terms are attached to a sulfur atom. Whenever a ring or ring system is substituted with an oxo group, the carbon atom to which the oxo is linked is a saturated carbon.

The radical Het is a heterocycle as specified in this specification and claims. Preferred amongst the Het radicals are those that are monocyclic.

Examples of Het comprise, for example, pyrrolidinyl, piperidinyl, morpholinyl, thiomorpholinyl, piperazinyl, pyrrolyl, pyrazolyl, imidazolyl, oxazolyl, isoxazolyl, thiazinolyl, isothiazinolyl, thiazolyl, isothiazolyl, oxadiazolyl, thiadiazolyl, triazolyl (including 1,2,3-triazolyl, 1,2,4-triazolyl), tetrazolyl, furanyl, thienyl, pyridyl, pyrimidyl, pyridazinyl, triazinyl, and the like. Of interest amongst the Het radicals are those which are non-saturated, in particular those having an aromatic character. Of further interest are those Het radicals having one or two nitrogens.

Each of the Het radicals mentioned in this and the following paragraph may be optionally substituted with the number and kind of substituents mentioned in the definitions of the compounds of formula (I) or any of the subgroups of compounds of formula (I). Some of the Het radicals mentioned in this and the following paragraph may be substituted with one, two or three hydroxy substituents. Such hydroxy substituted rings may occur as their tautomeric form's bearing keto groups. For example a 3-hydroxypyridazine moiety can occur in its tautomeric form 2H-pyridazin-3-one. Where Het is piperazinyl, it preferably is substituted in its 4-position by a substituent linked to the 4-nitrogen with a carbon atom, e.g. 4-C₁₋₆alkyl, 4-polyhaloC₁₋₆alkyl, C₁₋₆alkoxyC₁₋₆alkyl, C₁₋₆alkylcarbonyl, C₃₋₇cycloalkyl.

Interesting Het radicals comprise, for example pyrrolidinyl, piperidinyl, morpholinyl, thiomorpholinyl, piperazinyl, pyrrolyl, pyrazolyl, imidazolyl, oxazolyl, isoxazolyl, thiazolyl, isothiazolyl, oxadiazolyl, thiadiazolyl, triazolyl (including 1,2,3-triazolyl, 1,2,4-triazolyl), tetrazolyl, furanyl, thienyl, pyridyl, pyrimidyl, pyridazinyl, pyrazolyl, triazinyl, or any of such heterocycles condensed with a benzene ring, such as indolyl, indazolyl (in particular 1H-indazolyl), indolinyl, quinolinyl, tetrahydroquinolinyl (in particular 1,2,3,4-tetrahydroquinolinyl), isoquinolinyl, tetrahydroisoquinolinyl (in particular 1,2,3,4-tetrahydroisoquinolinyl), quinazolinyl, phthalazinyl, benzimidazolyl, benzoxazolyl, benzisoxazolyl, benzothiazolyl, benzoxadiazolyl, benzothiadiazolyl, benzofuranyl, benzothienyl.

The Het radicals pyrrolidinyl, piperidinyl, morpholinyl, thiomorpholinyl, piperazinyl, 4-substituted piperazinyl preferably are linked via their nitrogen atom (i.e. 1-pyrrolidinyl, 1-piperidinyl, 4-thiomorpholinyl, 4-morpholinyl, 1-piperazinyl, 4-substituted 1-piperazinyl).

It should be noted that the radical positions on any molecular moiety used in the definitions may be anywhere on such moiety as long as it is chemically stable.

“Heterocycle” as used herein includes by way of example and not limitation these heterocycles described in Paquette, Leo A.; Principles of Modern Heterocyclic Chemistry (W.A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S). Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 4H-indazoly, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

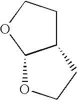

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” refers to a saturated, unsaturated or aromatic ring having up to about 25 carbon atoms. Typically, a carbocycle has about 3 to 7 carbon atoms as a monocycle, about 7 to 12 carbon atoms as a bicycle, and up to about 25 carbon atoms as a polycycle. Monocyclic carbocycles typically have 3 to 6 ring atoms, still more typically 5 or 6 ring atoms. Bicyclic carbocycles typically have 7 to 12 ring atoms, e.g., arranged as a bicyclo [4,5], [5,5], [5,6] or [6,6] system, or 9 or 10 ring atoms arranged as a bicyclo [5,6] or [6,6] system. The term carbocycle includes “cycloalkyl” which is a saturated or unsaturated carbocycle. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl.

The term “PRT” is selected from the terms “prodrug moiety” and “protecting group” as defined herein.

Radicals used in the definitions of the variables include all possible isomers unless otherwise indicated. For instance pyridyl includes 2-pyridyl, 3-pyridyl and 4-pyridyl; pentyl includes 1-pentyl, 2-pentyl and 3-pentyl.

Whenever a compound described herein is substituted with more than one of the same designated group, e.g., “R¹¹¹” or “A³”, then it will be understood that the groups may be the same or different, i.e., each group is independently selected.

By way of example and not limitation, A³, A² and R¹¹¹ are all recursive substituents in certain embodiments. Typically, each of these may independently occur 20, 19, 18, 17, 16, 15, 14, 13, 12, 11, 10, 9, 8, 7, 6, 5, 4, 3, 2, 1, or 0, times in a given embodiment. More typically, each of these may independently occur 12 or fewer times in a given embodiment. Whenever a compound described herein is substituted with more than one of the same designated group, e.g., “R¹¹¹” or “A³”, then it will be understood that the groups may be the same or different, i.e., each group is independently selected. Wavy lines indicate the site of covalent bond attachments to the adjoining groups, moieties, or atoms.

The entire content of International Patent Application Publication Number WO2007/014926 is hereby incorporated herein by reference. In particular, information relating to suitable synthetic routes for preparing the compounds of formulae (Ia) and (Ib), therein are hereby incorporated herein by reference.

When any variable occurs more than one time in any constituent, each definition is independent.

Whenever used hereinafter, the term “compounds of formula (I)”, or “the present compounds” or similar terms, it is meant to include the compounds of formula (I), their prodrugs, N-oxides, addition salts, quaternary amines, metal complexes, and stereochemically isomeric forms. One embodiment comprises the compounds of formula (I) or any subgroup of compounds of formula (I) specified herein, as well as the N-oxides, salts, as the possible stereoisomeric forms thereof. Another embodiment comprises the compounds of formula (I) or any subgroup of compounds of formula (I) specified herein, as well as the salts as the possible stereoisomeric forms thereof.

The compounds of formula (I) have several centers of chirality and exist as stereochemically isomeric forms. The term “stereochemically isomeric forms” as used herein defines all the possible compounds made up of the same atoms bonded by the same sequence of bonds but having different three-dimensional structures which are not interchangeable, which the compounds of formula (I) may possess.

With reference to the instances where (R) or (S) is used to designate the absolute configuration of a chiral atom within a substituent, the designation is done taking into consideration the whole compound and not the substituent in isolation.

Unless otherwise mentioned or indicated, the chemical designation of a compound encompasses the mixture of all possible stereochemically isomeric forms, which said compound may possess. Said mixture may contain all diastereomers and/or enantiomers of the basic molecular structure of said compound. All stereochemically isomeric forms of the compounds of the present invention both in pure form or mixed with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of the compounds of formula (I) or (II) can be obtained separately by conventional methods. Appropriate physical separation methods that may advantageously be employed are, for example, selective crystallization and chromatography, e.g. column chromatography.

For some of the compounds of formula (I) or (II), their prodrugs, N-oxides, salts, solvates, quaternary amines, or metal complexes, and the intermediates used in the preparation thereof, the absolute stereochemical configuration was not experimentally determined.

A person skilled in the art is able to determine the absolute configuration of such compounds using art-known methods such as, for example, X-ray diffraction.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

The term “prodrug” as used throughout this text means the pharmacologically acceptable derivatives such as esters, amides and phosphates, such that the resulting in vivo biotransformation product of the derivative is the active drug as defined in the compounds of formula (I) or (II). The reference by Goodman and Gilman (The Pharmacological Basis of Therapeutics, 8^{th} ed, McGraw-Hill, Int. Ed. 1992, “Biotransformation of Drugs”, p 13-15) describing prodrugs generally is hereby incorporated. Prodrugs preferably have excellent aqueous solubility, increased bioavailability and are readily metabolized into the active inhibitors in vivo. Prodrugs of a compound of the present invention may be prepared by modifying functional groups present in the compound in such a way that the modifications are cleaved, either by routine manipulation or in vivo, to the parent compound.

Preferred are pharmaceutically acceptable ester prodrugs that are hydrolysable in viva and are derived from those compounds of formula (I) or (II) having a hydroxy or a carboxyl group. An in viva hydrolysable ester is an ester, which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl which may be formed at any carboxy group in the compounds of this invention.

An in vivo hydrolysable ester of a compound of the formula (I) or (II) containing a hydroxy group includes inorganic esters such as phosphate esters and a-acyloxyalkyl ethers and related compounds which as a result of the in vivo hydrolysis of the ester breakdown to give the parent hydroxy group. Examples of a-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, salts of the compounds of formula (I) or (II) are those wherein the counter-ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable acid and base addition salts as mentioned hereinabove are meant to comprise the therapeutically active non-toxic acid and base addition salt forms which the compounds of formula (I) or (II) are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The compounds of formula (I) or (II) containing an acidicproton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds of formula (I) or (II) as well as the salts thereof, are able to form. Such solvates are for example hydrates, alcoholates and the like.

The term “quaternary amine” as used hereinbefore defines the quaternary ammonium salts which the compounds of formula (I) or (II) are able to form by reaction between a basic nitrogen of a compound of formula (I) or (II) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen.

Pharmaceutically acceptable counterions include chloro, bromo, iodo, trifluoroacetate and acetate. The counterion of choice can be introduced using ion exchange resins.

The N-oxide forms of the present compounds are meant to comprise the compounds of 5 formula (I) or (H) wherein one or several nitrogen atoms are oxidized to the so-called N-oxide.

It will be appreciated that the compounds of formula (I) or (II) may have metal binding, chelating, complex forming properties and therefore may exist as metal complexes or metal chelates. Such metalated derivatives of the compounds of formula (I) or (II) are intended to be included within the scope of the present invention.

Some of the compounds of formula (I) or (II) may also exist in their tautomeric form. Such forms although not explicitly indicated in the above formula are intended to be included within the scope of the present invention.

As mentioned above, the compounds of formula (I) or (II) have several asymmetric centers. In order to more efficiently refer to each of these asymmetric centers, the numbering system as indicated in the following representative structural formula will be used.

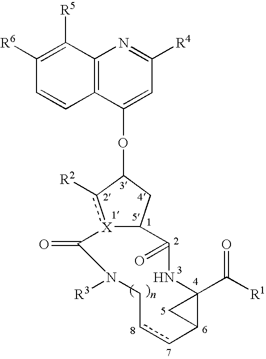

Asymmetric centers are present at positions 1, 4 and 6 of the macrocycle as well as at the carbon atom 3′ in the 5-membered ring, carbon atom 2′ when the R² substituent is C₁₋₆alkyl, and at carbon atom 1′ when. X is CH. Each of these asymmetric centers can occur in their R or S configuration.

The stereochemistry at position 1 preferably corresponds to that of an L-amino acid configuration, i.e. that of L-proline.

When X is CH, the 2 carbonyl groups substituted at positions 1′ and 5′ of the cyclopentane ring preferably are in a trans configuration. The carbonyl substituent at position 5′ preferably is in that configuration that corresponds to an L-proline configuration. The carbonyl groups substituted at positions 1′ and 5′ preferably are as depicted below in the structure of the following formula:

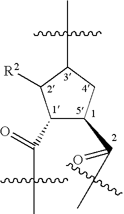

The compounds of formula (I) or (II) include a cyclopropyl group as represented in the structural fragment below:

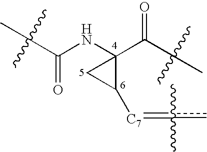

wherein C7 represents the carbon at position 7 and carbons at position 4 and 6 are asymmetric carbon atoms of the cyclopropane ring.

Notwithstanding other possible asymmetric centers at other segments of the compounds of formula (I) or (II), the presence of these two asymmetric centers means that the compounds can exist as mixtures of diastereomers, such as the diastereomers of compounds of formula (I) or (II) wherein the carbon at position 7 is configured either syn to the carbonyl or syn to the amide as shown below.

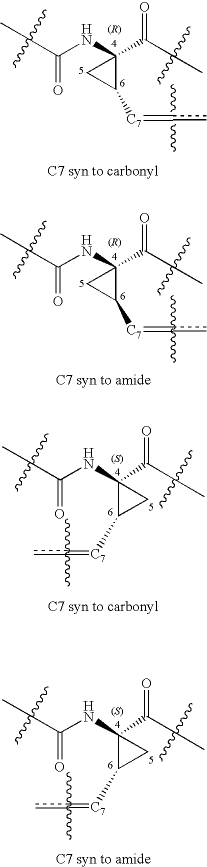

One embodiment concerns compounds of formula (I) or (II) wherein the carbon at position 7 is configured syn to the carbonyl. Another embodiment concerns compounds of formula (I) or (II) wherein the configuration at the carbon at position 4 is R. A specific subgroup of compounds of formula (I) or (II) are those wherein the carbon at position 7 is configured syn to the carbonyl and wherein the configuration at the carbon at position 4 is R.

The compounds of formula (I) or (II) may include as well a proline residue (when X is N) or a cyclopentyl or cyclopentenyl residue (when X is CH or C). Preferred are the compounds of formula (I) or (II) wherein the substituent at the 1 (or 5′) position and the substituent at position 3′ are in a trans configuration. Of particular interest are the compounds of formula (I) or (II) wherein position 1 has the configuration corresponding to L-proline and the substituent at position 3′ is in a trans configuration in respect of position 1. Preferably the compounds of formula (I) or (II) have the stereochemistry as indicated in the structures of formulae (I-a) and (I-b) below:

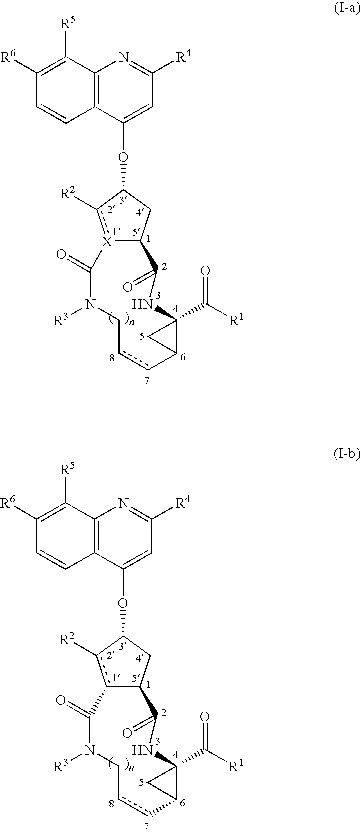

One embodiment of the present invention concerns compounds of formula (I) or (TI) or of formula (I-a) or of any subgroup of compounds of formula (I) or (II), wherein one or more of the following conditions apply:

(a) R² is hydrogen;
(b) X is nitrogen;
(c) a double bond is present between carbon atoms 7 and 8.

One embodiment of the present invention concerns compounds of formula (I) or (II) or of formulae (I-a), (I-b), or of any subgroup of compounds of formula (I) or (II), wherein one or more of the following conditions apply:

(a) R² is hydrogen;

### (b) X is CH;

(c) a double bond is present between carbon atoms 7 and 8.

Particular subgroups of compounds of formula (I) or (II) are those represented by the following structural formulae:

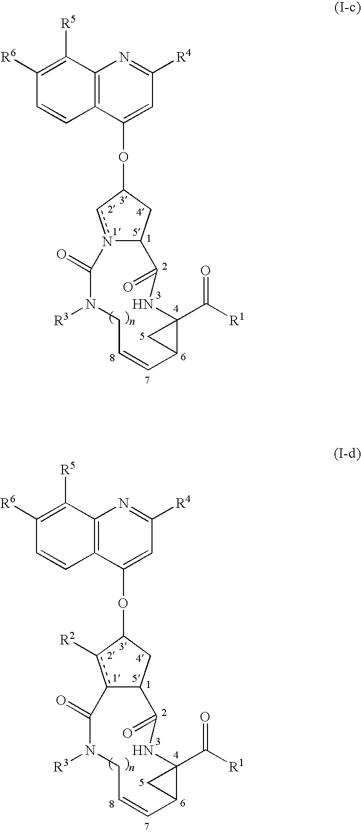

Amongst the compounds of formula (I-c) and (I-d), those having the stereochemical configuration of the compounds of formulae (I-a), and (I-b), respectively, are of particular interest.

The double bond between carbon atoms 7 and 8 in the compounds of formula (I) or (II), or in any subgroup of compounds of formula (I) or (II), maybe in a cis or in a trans configuration. Preferably the double bond between carbon atoms 7 and 8 is in a cis configuration, as depicted in formulae (I-c) and (I-d).

A double bond between carbon atoms 1′ and 2′ maybe present in the compounds of formula (I) or (II), or in any subgroup of compounds of formula (I) or (II), as depicted in formula (I-e) below.

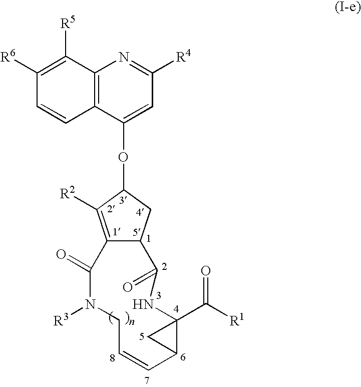

Yet another particular subgroup of compounds of formula (I) or (II) are those represented by the following structural formulae:

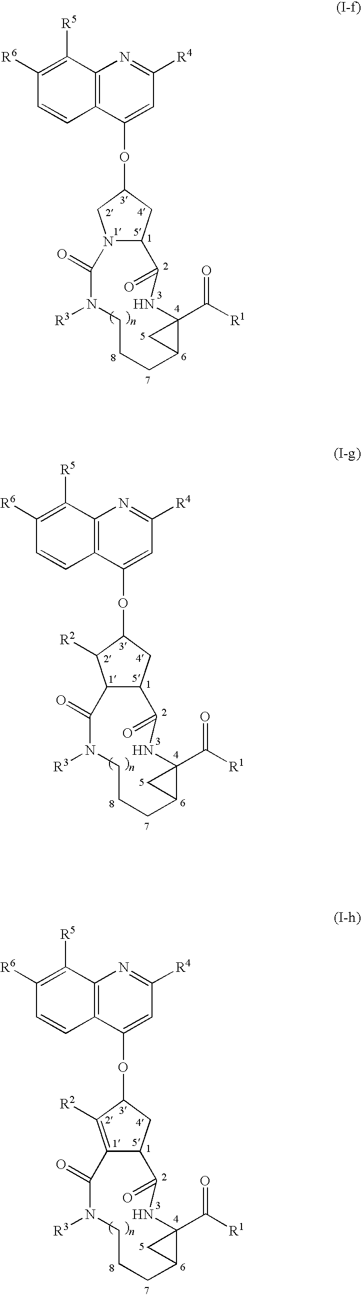

Amongst the compounds of formulae (I-f), (I-g) or (I-h), those having the stereochemical configuration of the compounds of formulae (I-a) and (I-b) are of particular interest.

In (I-a), (I-b), (I-c), (I-d), (I-e), (I-f), (I-g) and (I-h), where applicable, X, n, R¹, R², R³, R⁴, R⁵, and R⁶ are as specified in the definitions of the compounds of formula (I) or (II) or in any of the subgroups of compounds of formula (I) or (II) specified herein.

It is to be understood that the above defined subgroups of compounds of formulae (I-a), (I-b), (I-c), (I-d), (I-e), (I-f), (I-g) or (I-h), as well as any other subgroup defined herein, are meant to also comprise any N-oxides, addition salts, quaternary amines, metal complexes and stereochemically isomeric forms of such compounds.

When n is 2, the moiety —CH₂— bracketed by “n” corresponds to ethanediyl in the compounds of formula (I) or (II) or in any subgroup of compounds of formula (I) or (II). When n is 3, the moiety —CH₂— bracketed by “n” corresponds to propanediyl in the compounds of formula (I) or (II) or in any subgroup of compounds of formula (I) or (II). When n is 4, the moiety —CH₂— bracketed by “n” corresponds to butanediyl in the compounds of formula (I) or (II) or in any subgroup of compounds of formula (I) or (II). When n is 5, the moiety —CH₂ bracketed by “n” corresponds to pentanediyl in the compounds of formula (I) or (II) or in any subgroup of compounds of formula (I) or (II). When n is 6, the moiety —CH₂— bracketed by “n” corresponds to hexanediyl in the compounds of formula (I) or (II) or in any subgroup of compounds of formula (I) or (II). Particular subgroups of the compounds of formula (I) or (II) are those compounds wherein n is 4 or 5.

Embodiments of the invention are compounds of formula (I) or (II) or any of the subgroups of compounds of formula (I) or (II).

In a specific embodiment of the invention R^{f} is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;

each Rₕ and Rᵢ is independently H, alkyl, or haloalkyl; and R_{d} and Rₑ are each independently H, (C1-10)alkyl, or aryl, which is optionally substituted with one or more halo;

In a specific embodiment of the invention R^{f} is alkyl, aryl, cycloalkyl, which R^{f} is optionally substituted with one or more R^{g} independently selected from alkyl, halo, —C(═O)OR_{d}, or trifluoromethyl, wherein each alkyl of R^{g} is optionally substituted with one or more halo, alkoxy, or cyano.

In a specific embodiment of the invention R^{f} is aryl, heteroaryl, or cycloalkyl, which R^{f} is optionally substituted with one to three A³.

In a specific embodiment of the invention R^{f} is cyclopropyl which R^{f} is optionally substituted by up to four A³.

In a specific embodiment of the invention R^{f} is cyclopropyl which R^{f} is optionally substituted by one A³.

In a specific embodiment of the invention W is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo or cyano; and

each Rₕ and Rᵢ is independently H, alkyl, or haloalkyl.

In a specific embodiment of the invention R^{f} is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;

each Rₕ and Rᵢ is independently H, alkyl, or haloalkyl;

In a specific embodiment of the invention R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

In a specific embodiment of the invention R^{f} is cyclopropyl.

In a specific embodiment of the invention R^{f} is 1-methylcyclopropyl.

In a specific embodiment of the invention for compounds of formula (I) or (II) or any of the subgroups of compounds of formula (I) or (II) wherein carbon atoms bearing four substituents and including at least one bond to hydrogen in a compound of structure (I) or (II) may optionally have one or more of their hydrogen atoms replaced by halo where the halo can be F, Cl, Br or I, preferably F.

Further embodiments of the invention are compounds of formula (I) or (II) or any of the subgroups of compounds of formula (I) or (II) wherein
(a) R² is hydrogen;
(b) R² is C₁₋₆ alkyl, preferably methyl.

Embodiments of the invention are compounds of formula (I) or (II) or any of the subgroups of compounds of formula (I) or (II) wherein
(a) X is N, C (X being linked via a double bond) or CH (X being linked via a single bond) and R² is hydrogen;
(b) X is C (X being linked via a double bond) and R² is C₁₋₆alkyl, preferably methyl.

Further embodiments of the invention are compounds of formula (I) or (II) or any of the subgroups of compounds of formula (I) or (II) wherein
(a) R³ is hydrogen;
(b) R³ is C₁₋₆alkyl;
(c) R³ is C₁₋₆alkoxyC₁₋₆alkyl or C₃₋₇cycloalkyl.

Preferred embodiments of the invention are compounds of formula (I) or (II) or any of the subgroups of compounds of formula (I) or (II) wherein R³ is hydrogen, or C₁₋₆ alkyl, more preferably hydrogen or methyl.

Embodiments of the invention are compounds of formula (I) or (II) or any of the subgroups of compounds of formula (I) or (II) wherein R⁴ is aryl or Het, each independently, optionally substituted with any of the substituents of Het or aryl mentioned in the definitions of the compounds of formula (I) or (II) or of any of the subgroups of compounds of formula (I) or (II); or specifically said aryl or Het being each, independently, optionally substituted with C₁₋₆alkyl, halo, amino, mono- or di-C₁₋₆alkylamino, pyrrolidinyl, piperidinyl, morpholinyl, piperazinyl, 4-C₁₋₆alkylpiperazinyl; and wherein the morpholinyl and piperidinyl groups may optionally substituted with one or two C₁₋₆ alkyl radicals;

Embodiments of the invention are compounds of formula (I) or (II) or any of the subgroups of compounds of formula (I) or (II) wherein R⁴ is a radical

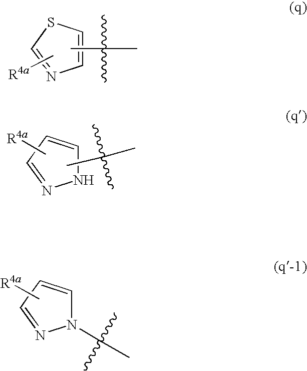

or, in particular, wherein R⁴ is selected from the group consisting of:

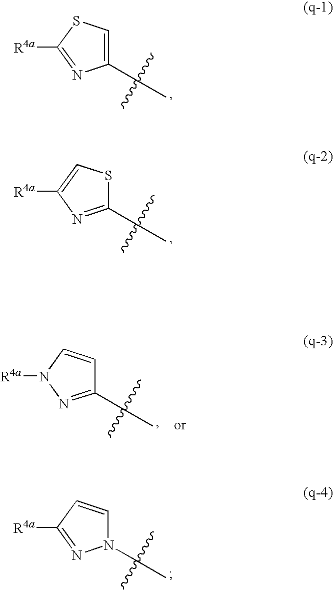

wherein, where possible a nitrogen may bear an R^{4a} substituent or a link to the remainder of the molecule; each R^{4a} in any of the R⁴ substituents may be selected from those mentioned as possible substituents on Het, as specified in the definitions of the compounds of formula (I) or (II) or of any of the subgroups of compounds of formula (I) or (II);

more specifically each R^{4a} may be hydrogen, halo, C₁₋₆alkyl, amino, or mono- or di-C₁₋₆alkylamino, pyrrolidinyl, piperidinyl, morpholinyl, piperazinyl, 4-C₁₋₆alkyl-piperazinyl; and wherein the morpholinyl and piperidinyl groups may optionally substituted with one or two C₁₋₆alkyl radicals;

more specifically each R^{4a} is, each independently, hydrogen, halo, C₁₋₆alkyl, amino, or mono- or di-C₁₋₆alkylamino;

and where R^{4a} is substituted on a nitrogen atom, it preferably is a carbon containing 5 substituent that is connected to the nitrogen via a carbon atom or one of its carbon atoms; and wherein in that instance R^{4a} preferably is C₁₋₆alkyl.

Embodiments of the invention are compounds of formula (I) or (II) or any of the subgroups of compounds of formula (I) or (II) wherein R⁴ is phenyl or pyridiyl (in particular 4-pyridyl) which each may be substituted with 1, 2 or 3 substituents selected from those mentioned for aryl in the definitions of the compounds of formula (I) or (II) or of any of the subgroups thereof. In particular said phenyl or pyridyl is substituted with 1-3 (or with 1-2, or with one) substituent or substituents selected from halo, C₁₋₆alkyl or C₁₋₆alkoxy.

Embodiments of the invention are compounds of formula (I) or (II) or any of the subgroups of compounds of formula (I) or (II) wherein R⁵ is halo, or C₁₋₆alkyl, preferably methyl, ethyl, isopropyl, tert-butyl, fluoro, chloro, or bromo include polyhaloC₁₋₆alkyl

Embodiments of the invention are compounds of formula (I) or (II) or any of the subgroups of compounds of formula (I) wherein R⁶ is C₁₋₆alkoxy or alkylamino; preferably R⁶ is methoxy or dimethylamino; more preferably R⁶ is methoxy.

The compounds of formula (I) or (II) consist of three building blocks P1, P2, P3. Building block P1 further contains a P1′ tail. The carbonyl group marked with an asterisk in compound (I-c) below may be part of either building block P2 or of building block P3. For reasons of chemistry, building block P2 of the compounds of formula (I) wherein X is C incorporates the carbonyl group attached to the position 1′.

The linking of building blocks P1 with P2, P2 with P3, and P1 with P1′ (when R¹ is —NH—SO₃R^{f}) involves forming an amide bond. The linking of blocks P1 and P3 involves double bond formation. The linking of building blocks P1, P2 and P3 to prepare compounds (I-i) or (I-j) can be done in any given sequence. One of the steps involves a cyclization whereby the macrocycle is formed.

Represented herebelow are compounds (I-i) which are compounds of formula (I) or (II) wherein carbon atoms C7 and C8 are linked by a double bond, and compounds (I-j) which are compounds of formula (I) or (II) wherein carbon atoms C7 and C8 are linked by a single bond. The compounds of formula (I-j) can be prepared from the corresponding compounds of formula (I-i) by reducing the double bond in the macrocycle.

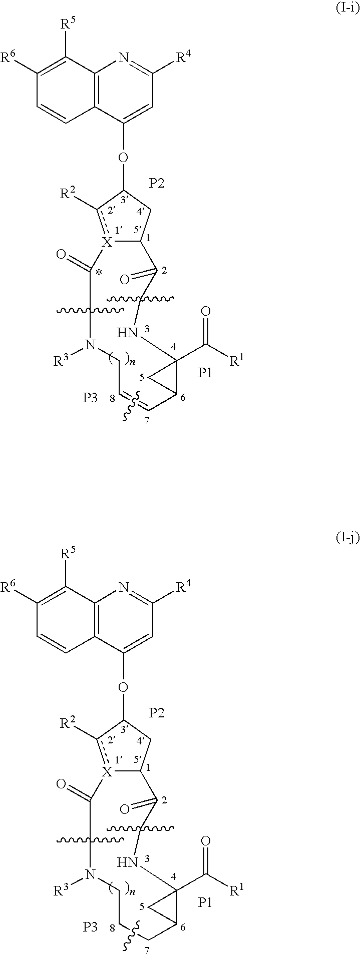

It should be noted that in compounds of formula (I-c), the amide bond formation between blocks P2 and P3 may be accomplished at two different positions of the urea fragment. A first amide bond encompasses the nitrogen of the pyrrolidine ring and the adjacent carbonyl (marked with an asterisk). An alternative second amide bond formation involves the reaction of the asterisked carbonyl with a —NHR³ group. Both amide bond formations between building blocks P2 and P3 are feasible.

The synthesis procedures described hereinafter are meant to be applicable for as well the racemates, stereochemically pure intermediates or end products, or any stereoisomeric mixtures. The racemates or stereochemical mixtures may be separated into stereoisomeric forms at any stage of the synthesis procedures. In one embodiment, the intermediates and end products have the stereochemistry specified above in the compounds of formula (I-a) and (I-b).

In order to simplify the structural representation of the compounds of formula (I) or (II) or the intermediates the group

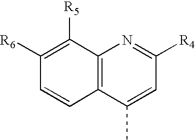

or the group Het¹ is represented by R⁹ and the dotted line represents the bond linking said group represented by R⁹ to the remainder of the molecule.

In another embodiment R⁹ is Het¹.

A specific embodiment concerns the following compound and its salts and compounds where the amino group is protected by a protecting group:

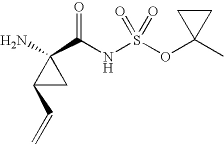

A specific embodiment concerns the following compound:

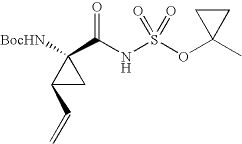

A specific embodiment concerns the following compound and its salts and compounds where the amino group is protected by a protecting group:

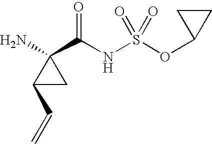

A specific embodiment concerns the following compound:

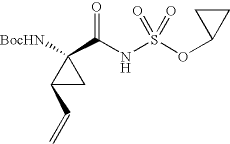

A specific embodiment concerns the following compound and its salts and compounds where the amino group is protected by a protecting group:

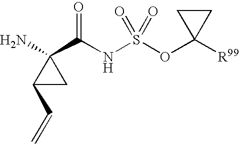

In one embodiment, compounds (I-i) are prepared by first forming the amide bonds and 5 subsequent forming the double bond linkage between P3 and P1 with concomitant cyclization to the macrocycle. In a preferred embodiment, compounds (1) or (II) wherein the bond between C7 and C8 is a double bond, which are compounds of formula (I-i), as defined above, may be prepared as outlined in the following reaction scheme:

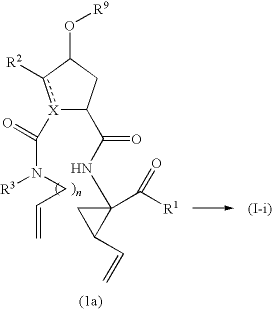

Formation of the macrocycle can be carried out via an olefin metathesis reaction in the presence of a suitable metal catalyst such as e.g. the Ru-based catalyst reported by Miller, S. J., Blackwell, 11.E., Grubbs, R H. J. Am. Chem. Soc. 118, (1996), 9606-9614; Kingsbury, J. S., Harrity, J. P. A., Bonitatebus, P. J., Hoveyda, A. H., J. Am. Chem. Soc. 121, (1999), 791-799; and Huang et al., J. Am. Chem. Soc. 121, (1999), 26742678; for example a Hoveyda-Grubbs catalyst.

Air-stable ruthenium catalysts such as bis(tricyclohexylphosphine)-3-phenyl-1H-inden-1-ylidene ruthenium chloride (Neolyst M1®) or bis(tricyclohexylphosphine)-[(phenylthio)methyleneruthenium (IV) dichloride can be used. Other catalysts that can be used are Grubbs first and second generation catalysts, i.e. Benzylidene-bis(tricyclehexylphosphine)dichlororuthenium and (1,3-bis-(2,4,6-trimethylphenyl)-2-imidazolidinylidene)dichloro(phenylmethylene)-(tricyclohexylphosphine)ruthenium, respectively. Of particular interest are the Hoveyda-Grubbs first and second generation catalysts, which are dichloro(o-isopropoxyphenylmethylene)(tricyclohexylphosphine)-ruthenium(II) and 1,3-bis-(2,4,6-trimethylphenyl)-2-imidazolidinylidene)dichloro-(o-isopropoxyphenylmethylene)ruthenium respectively. Also other catalysts containing other transition metals such as Mo can be used for this reaction. The metathesis reactions may be conducted in a suitable solvent such as for example ethers, e.g. THF, dioxane; halogenated hydrocarbons, e.g. dichloromethane, CHCl₃, 1,2-dichloroethane and the like, hydrocarbons, e.g. toluene. In a preferred embodiment, the metathesis reaction is conducted in toluene. These reactions are conducted at increased temperatures under nitrogen atmosphere.

Compounds of formula (I) or (II) wherein the link between C7 and C8 in the macrocycle is a single bond, i.e. compounds of formula (I-j), can be prepared from the compounds of formula (I-i) by a reduction of the C7-C8 double bond in the compounds of formula (I-i). This reduction may be conducted by catalytic hydrogenation with hydrogen in the presence of a noble metal catalyst such as, for example, Pt, Pd, Rh, Ru or Raney nickel. Of interest is Rh on alumina. The hydrogenation reaction preferably is conducted in a solvent such as, e.g. an alcohol such as methanol, ethanol, or an ether such as THF, or mixtures thereof. Water can also be added to these solvents or solvent mixtures

The R¹ group can be connected to the P1 building block at any stage of the synthesis, i.e. before or after the cyclization, or before or after the cyclization and reduction as described herein above. The compounds of formula (I) or (II) wherein R¹ represents —NHSO₃R^{f}, said compounds being represented by formula (I-k-1), can be prepared by linking the R¹ group to P1 by forming an amide bond between both moieties. In one embodiment —NHSO₃R^{f} groups are introduced in the last step of the synthesis of the compounds (I) or (II) as outlined in the following reaction schemes wherein G represents a group:

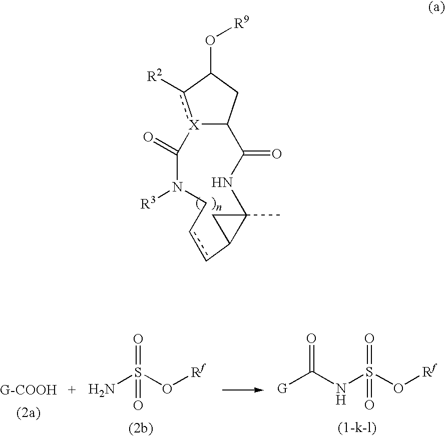

Intermediate (2a) can be coupled with the amine (2b) by an amide forming reaction such as any of the procedures for the formation of an amide bond described hereinafter. In particular, (2a) may be treated with a coupling agent, for example N,N′-carbonyl-diimidazole (CDT), EEDQ, IIDQ, EDCI or benzotriazol-1-yl-oxy-tris-pyrrolidino-phosphonium hexafluorophosphate (commercially available as PyBOP®), or O-(7-azabenzotriazol-1-yl)-N,N,N′,N′-tetramethyluronium hexafluorophosphate (HATU), in a solvent such as an ether, e.g. THE, or a halogenated hydrocarbon, e.g. dichloromethane, chloroform, dichloroethane, and reacted with the desired sulfamate (2b), preferably after reacting (2a) with the coupling agent. The reactions of (2a) with (2b) preferably are conducted in the presence of a base, for example a trialkylamine such as triethylamine or diisopropylethylamine, or 1,8-diazabicycle[5.4.0]undec-7-ene (DBU). Intermediate (2a) can also be converted into an activated form, e.g. an activated form of general formula G-CO-Z, wherein Z represents halo, or the rest of an active ester, e.g. Z is an aryloxy group such as phenoxy, p.nitrophenoxy, pentafluorophenoxy, trichlorophenoxy, pentachlorophenoxy and the like; or Z can be the rest of a mixed anhydride. In one embodiment, G-CO-Z is an acid chloride (G-CO—Cl) or a mixed acid anhydride (G-CO—O—CO—R or G-CO—O—CO—OR, R in the latter being e.g. C₁₋₄alkyl, such as methyl, ethyl, propyl, i.propyl, butyl, t.butyl, i.butyl, or benzyl). The activated form G-CO-Z is reacted with the sulfamate (2b).

The activation of the carboxylic acid in (2a) as described in the above reactions may lead to an internal cyclization reaction to an azalactone intermediate of formula:

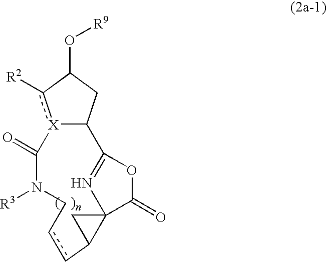

wherein X, R², R³, R⁹, n are as specified above and wherein the stereogenic centers may have the stereochemical configuration as specified above, for example as in (I-a) or (I-b). The intermediates (2a-1) can be isolated from the reaction mixture, using conventional methodology, and the isolated intermediate (2a-1) is then reacted with (2b), or the reaction mixture containing (2a-1) can be reacted further with (2b) without isolation of (2a-1). In one embodiment, where the reaction with the coupling agent is conducted in a water-immiscible solvent, the reaction mixture containing (2a-1) may be washed with water or with slightly basic water in order to remove all water-soluble side products. The thus obtained washed solution may then be reacted with (2b) without additional purification steps. The isolation of intermediates (2a-1) on the other hand may provide certain advantages in that the isolated product, after optional further purification, may be reacted with (2b), giving rise to less side products and an easier work-up of the reaction.

Intermediate (2a) can be coupled with the alcohol (2c) by an ester forming reaction. For example, (2a) and (2c) are reacted together with removal of water either physically, e.g. by azeotropical water removal, or chemically by using a dehydrating agent. Intermediate (2a) can also be converted into an activated form G-CO-Z, such as the activated forms mentioned above, and subsequently reacted with the alcohol (2c). The ester forming reactions preferably are conducted in the presence of a base such as an alkali metal carbonate or hydrogen carbonate, e.g. sodium or potassium hydrogen carbonate, or a tertiary amine such as the amines mentioned herein in relation to the amide forming reactions, in particular a trialkylamine, e.g. triethylamine. Solvents that can be used in the ester forming reactions comprise ethers such as THF; halogenated hydrocarbons such as dichloromethane, CH₂Cl₂; hydrocarbons such as toluene; polar aprotic solvents such as DMF, DMSO, DMA; and the like solvents.

The compounds of formula (I) or (II) wherein R³ is hydrogen, said compounds being represented by (I-1), can also be prepared by removal of a protecting group PG, from a corresponding nitrogen-protected intermediate (3a), as in the following reaction scheme. The protecting group PG in particular is any of the nitrogen protecting groups mentioned hereinafter and can be removed using procedures also mentioned hereinafter:

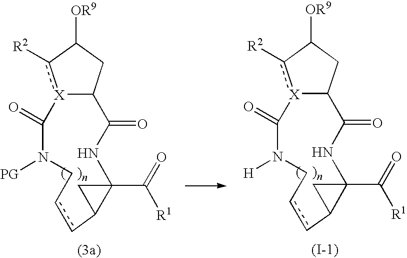

The starting materials (3a) in the above reaction can be prepared following the procedures for the preparation of compounds of formula (I) or (11), but using intermediates wherein the group R³ is PG.

The compounds of formula (I) or (II) can also be prepared by reacting an intermediate (4a) with intermediate (4b) as outlined in the following reaction scheme wherein the various radicals have the meanings specified above:

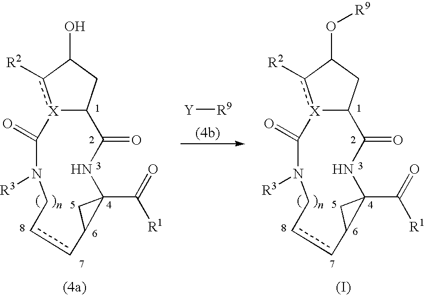

Y in (4b) represents hydroxy or a leaving group LG such as a halide, e.g. bromide or chloride, or an arylsulfonyl group, e.g. mesylate, triflate or tosylate and the like.

In one embodiment, the reaction of (4a) with (4b) is an o-arylation reaction and Y represents a leaving group. This reaction can be conducted following the procedures described by E. M. Smith et al. (J. Med. Chem. (1988), 31, 875-885). In particular, this reaction is conducted in the presence of a base, preferably a strong base, in a reaction-inert solvent, e.g. one of the solvents mentioned for the formation of an amide bond.

In a particular embodiment, starting material (4a) is reacted with (4b) in the presence of a base which is strong enough to detract a hydrogen from the hydroxy group, for example an alkali of alkaline metal hydride such as LiH or sodium hydride, or alkali metal alkoxide such as sodium or potassium methoxide or ethoxide, potassium tert-butoxide, in a reaction inert solvent like a dipolar aprotic solvent, e.g. DMA, DMF and the like. The resulting alcoholate is reacted with the arylating agent (4b), wherein Y is a suitable leaving group as mentioned above. The conversion of (4a) to (I) or (II) using this type of O-arylation reaction does not change the stereochemical configuration at the carbon bearing the hydroxy group.

Alternatively, the reaction of (4a) with (4b) can also be conducted via a Mitsunobu reaction (Mitsunobu, 1981, Synthesis, January, 1-28; Rano et al., Tetrahedron Let, 1995, 36, 22, 3779-3792; Krchnak et al., Tetrahedron Lett, 1995, 36, 5, 6193-6196; Richter et al, Tetrahedron Lett., 1994, 35, 27, 4705-4706). This reaction comprises treatment of intermediate (4a) with (4b) wherein Y is hydroxyl, in the presence of triphenylphosphine and an activating agent such as a dialkyl azocarboxylate, e.g. diethyl azodicarboxylate (DEAD), diisopropyl azodicarboxylate (DIAD) or the like. The Mitsunobu reaction changes the stereochemical configuration at the carbon bearing the hydroxy group.

Alternatively, in order to prepare the compounds of formula (I), first an amide bond between building blocks P2 and P1 is formed, followed by coupling of the P3 building block to the P1 moiety in P1-P2, and a subsequent carbamate or ester bond formation between P3 and the P2 moiety in P2-P1-P3 with concomitant ring closure.

Yet another alternative synthetic methodology is the formation of an amide bond between building blocks P2 and P3, followed by the coupling of building block P1 to the P3 moiety in P3-P2, and a last amide bond formation between P1 and P2 in P1-P3-P2 with concomitant ring closure.

Building blocks P1 and P3 can be linked to a P1-P3 sequence. If desired, the double bond linking P1 and P3 may be reduced. The thus formed P1-P3 sequence, either reduced or not, can be coupled to building block P2 and the thus forming sequence P1-P3-P2 subsequently cyclized, by forming an amide bond.

Building blocks P1 and P3 in any of the previous approaches can be linked via double bond formation, e.g. by the olefin metathesis reaction described hereinafter, or a Wittig type reaction. If desired, the thus formed double bond can be reduced, similarly as described above for the conversion of (I-i) to (I-j). The double bond can also be reduced at a later stage, i.e. after addition of a third building block, or after formation of the macrocycle. Building blocks P2 and P1 are linked by amide bond formation and P3 and P2 are linked by carbamate or ester formation.

The tail P1′ can be bonded to the P1 building block at any stage of the synthesis of the compounds of formula (I), for example before or after coupling the building blocks P2 and P1; before or after coupling the P3 building block to P1; or before or after ring closure.

The individual building blocks can first be prepared and subsequently coupled together or alternatively, precursors of the building blocks can be coupled together and modified at a later stage to the desired molecular composition.

The functionalities in each of the building blocks may be protected to avoid side reactions.

The formation of amide bonds can be carried out using standard procedures such as those used for coupling amino acids in peptide synthesis. The latter involves the dehydrative coupling of a carboxyl group of one reactant with an amino group of the other reactant to form a linking amide bond. The amide bond formation may be performed by reacting the starting materials in the presence of a coupling agent or by converting the carboxyl functionality into an active form such as an active ester, mixed anhydride or a carboxyl acid chloride or bromide. General descriptions of such coupling reactions and the reagents used therein can be found in general textbooks on peptide chemistry, for example, M. Bodanszky, “Peptide Chemistry”, 2nd rev. ed. 25 Springer-Verlag, Berlin, Germany, (1993).

Examples of coupling reactions with amide bond formation include the azide method, mixed carbonic-carboxylic acid anhydride (isobutyl chloroformate) method, the carbodiimide (dicyclohexylcarbodiimide, diisopropylcarbodiimide, or water-soluble carbodiimide such as N-ethylN′-[(3-dimethylamino)propyl]carbodiimide) method, the active ester method (e.g. p-nitrophenyl, p-chlorophenyl, trichlorophenyl, pentachlorophenyl, pentafluorophenyl, N-hydroxysuccinic imido and the like esters), the Woodward reagent K-method, the 1,1-carbonyldiimidazole (CDI or N,N′-carbonyldiimidazole) method, the phosphorus reagents or oxidation-reduction methods. Some of these methods can be enhanced by adding suitable catalysts, e.g. in the carbodiimide method by adding 1-hydroxybenzotriazole, DBU (1,8-diazabicyclo[5.4.0]undec-7-ene), or 4-DMAP. Further coupling agents are (benzotriazol-1-yloxy)tris-(dimethylamino) phosphonium hexafluorophosphate, either by itself or in the presence of 1-hydroxy-benzotriazole or 4-DMAP; or 2-(1H-benzotriazol-1-yl)-N,N,N′,N′-tetramethyluronium tetrafluoroborate, or 0-(7-azabenzotriazol-1-yl)-N,N,N′N′-tetramethyl-uronium hexafluorophosphate. These coupling reactions can be performed in either solution (liquid phase) or solid phase.

A preferred amide bond formation is performed employing N-ethyloxycarbonyl-2-ethyloxy-1,2-dihydroquinoline (EEDQ) or N-isobutyloxy-carbonyl-2-isobutyloxy-1,2-dihydroquinoline (IIDQ). Unlike the classical anhydride procedure, EEDQ and IIDQ do not require base nor low reaction temperatures. Typically, the procedure involves reacting equimolar amounts of the carboxyl and amine components in an organic solvent (a wide variety of solvents can be used). Then EEDQ or IIDQ is added in excess and the mixture is allowed to stir at room temperature.

The coupling reactions preferably are conducted in an inert solvent, such as halogenated hydrocarbons, e.g. dichloromethane, chloroform, dipolar aprotic solvents such as acetonitrile, dimethylformamide, dimethylacetamide, DMSO, IIMPT, ethers such as tetrahydrofuran (THF).

In many instances the coupling reactions are done in the presence of a suitable base such as a tertiary amine, e.g. triethylamine, diisopropylethylamine (DIPEA), N-methylmorpholine, N-methylpyrrolidine, 4-DMAP or 1,8-diazabicyclo[5.4.0]undec-7-ene (DBU). The reaction temperature may range between 0° C. and 50° C. and the reaction time may range between 15 min and 24 h.

The functional groups in the building blocks that are linked together may be protected to avoid formation of undesired bonds. Appropriate protecting groups that can be used are listed for example in Greene, “Protective Groups in Organic Chemistry”, John Wiley & Sons, New York (1999) and “The Peptides: Analysis, Synthesis, Biology”, Vol. 3, Academic Press, New York (1987).

Carboxyl groups can be protected as an ester that can be cleaved off to give the carboxylic acid.

Protecting groups that can be used include 1) alkyl esters such as methyl, trimethylsilyl and tertbutyl; 2) arylalkyl esters such as benzyl and substituted benzyl; or 3) esters that can be cleaved by a mild base or mild reductive means such as trichloroethyl and phenacyl esters.

Amino groups can be protected by a variety of N-protecting groups, such as:
1) acyl groups such as formyl, trifluoroacetyl, phthalyl, and p-toluenesulfonyl;
2) aromatic carbamate groups such as benzyloxycarbonyl (Cbz or Z) and substituted benzyloxycarbonyls, and 9-fluorenylmethyloxycarbonyl (Fmoc);
3) aliphatic carbamate groups such as tert-butyloxycarbonyl (Bac), ethoxycarbonyl, diisopropylmethoxy-carbonyl, and allyloxycarbonyl;
4) cyclic alkyl carbamate groups such as cyclopentyloxycarbonyl and adamantyloxycarbonyl;
5) alkyl groups such as triphenylmethyl, benzyl or substituted benzyl such as 4-methoxybenzyl;
6) trialkylsilyl such as trimethylsilyl or t.Bu dimethylsilyl; and
7) thiol containing groups such as phenylthiocarbonyl and dithiasuccinoyl. Interesting amino protecting groups are Boc and Fmoc.

Preferably the amino protecting group is cleaved off prior to the next coupling step. Removal of N-protecting groups can be done following art-known procedures. When the Boc group is used, the methods of choice are trifluoroacetic acid, neat or in dichloromethane, or Ha in dioxane or in ethyl acetate. The resulting ammonium salt is then neutralized either prior to the coupling or in situ with basic solutions such as aqueous buffers, or tertiary amines in dichloromethane or acetonitrile or dimethylformamide. When the Fmoc group is used, the reagents of choice are piperidine or substituted piperidine in dimethylformamide, but any secondary amine can be used. The deprotection is carried out at a temperature between 0° C. and room temperature, usually around 15-25° C., or 20-22° C.

Other functional groups that can interfere in the coupling reactions of the building blocks may also be protected. For example hydroxyl groups may be protected as benzyl or substituted benzyl ethers, e.g. 4-methoxybenzyl ether, benzoyl or substituted benzoyl esters, e.g. 4-nitrobenzoyl ester, or with trialkylsilyl groups (e.g. trimethylsilyl or tert-butyldimethylsilyl).

Further amino groups may be protected by protecting groups that can be cleaved off selectively. For example, when Boc is used as the a-amino protecting group, the following side chain protecting groups are suitable: p-toluenesulfonyl (tosyl) moieties can be used to protect further amino groups; benzyl (Bn) ethers can be used to protect hydroxy groups; and benzyl esters can be used to protect further carboxyl groups. Or when Fmoc is chosen for the a-amino protection, usually tert-butyl based protecting groups are acceptable. For instance, Boc can be used for further amino groups; tertbutyl ethers for hydroxyl groups; and tert-butyl esters for further carboxyl groups.

Any of the protecting groups may be removed at any stage of the synthesis procedure but preferably, the protecting groups of any of the functionalities not involved in the reaction steps are removed after completion of the build-up of the macrocycle. Removal of the protecting groups can be done in whatever manner is dictated by the choice of protecting groups, which manners are well known to those skilled in the art.

The intermediates of formula (Ia) wherein X is N, said intermediates being represented by formula (1a-1), may be prepared starting from intermediates (5a) which are reacted with an alkenamine (5b) in the presence of a carbonyl introducing agent as outlined in the following reaction scheme.

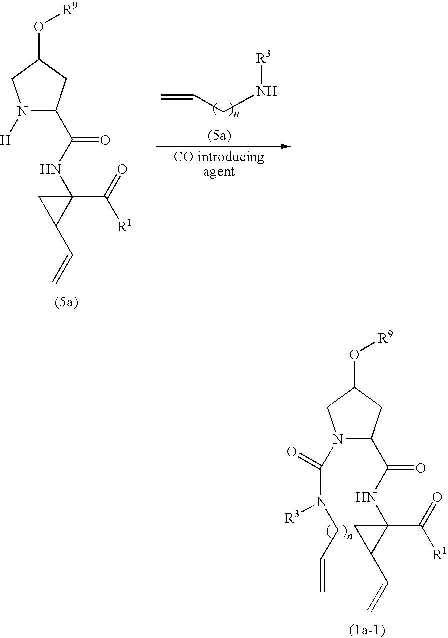

Carbonyl (CO) introducing agents include phosgene, or phosgene derivatives such as carbonyl diimidazole (CDI), and the like. In one embodiment (5a) is reacted with the CO introducing agent in the presence of a suitable base and a solvent, which can be the bases and solvents used in the amide forming reactions as described above. In a particular embodiment, the base is a hydrogencarbonate, e.g. NaHCO₃, or a tertiary amine such as triethylamine and the like, and the solvent is an ether or halogenated hydrocarbon, e.g. THF, CH₇Cl₂, CHCl₃, and the like. Thereafter, the amine (5b) is added thereby obtaining intermediates (1a-1) as in the above scheme. An alternative route using similar reaction conditions involves first reacting the CO introducing agent with the alkenamine (5b) and then reacting thus formed intermediate with (5a).

The intermediates (1a-1) can alternatively be prepared as follows:

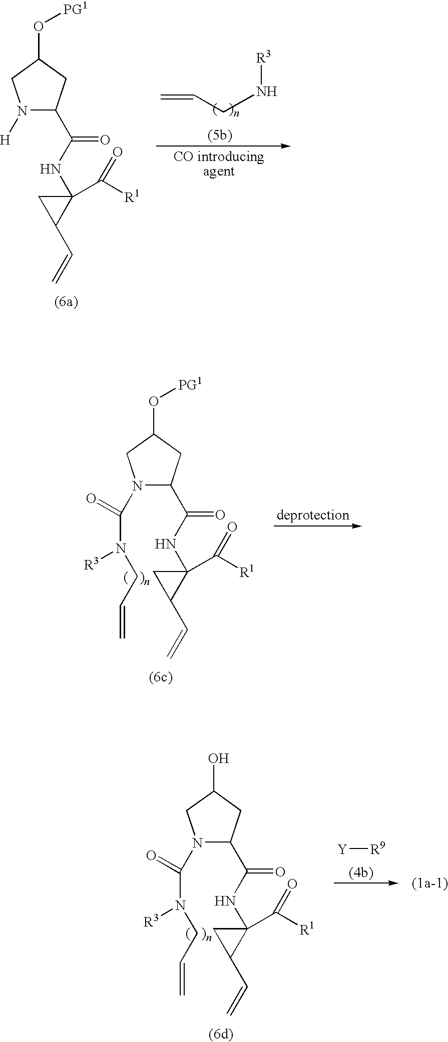

PG¹ is an O-protecting group, which can be any of the groups mentioned herein and in particular is a benzoyl or substituted benzoyl group such as 4-nitrobenzoyl. In the latter instance this group can be removed by reaction with an alkali metal hydroxide (LiOH, NaOH, KOH), in particular where PG¹ is 4-nitrobenzoyl, with LiOH, in an aqueous medium comprising water and a water-soluble organic solvent such as an alkanol (methanol, ethanol) and THF.

Intermediates (6a) are reacted with (b) in the presence of a carbonyl introducing agent, similar as described above, and this reaction yields intermediates (6c). These are deprotected, in particular using the reaction conditions mentioned above. The resulting alcohol (6d) is reacted with intermediates (4b) as described above for the reaction of (4a) with (4b) and this reaction results in intermediates (1a-1).

The intermediates of formula (1a) wherein X is C, said intermediates being represented by formula (1a-2), may be prepared by an amide forming reaction starting from intermediates (7a) which are reacted with an amine (b) as shown in the following reaction scheme, using reaction conditions for preparing amides such as those described above.

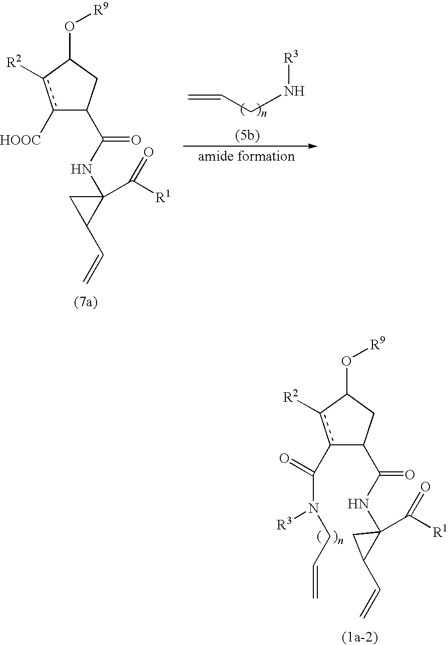

The intermediates (1a-1) can alternatively be prepared as follows:

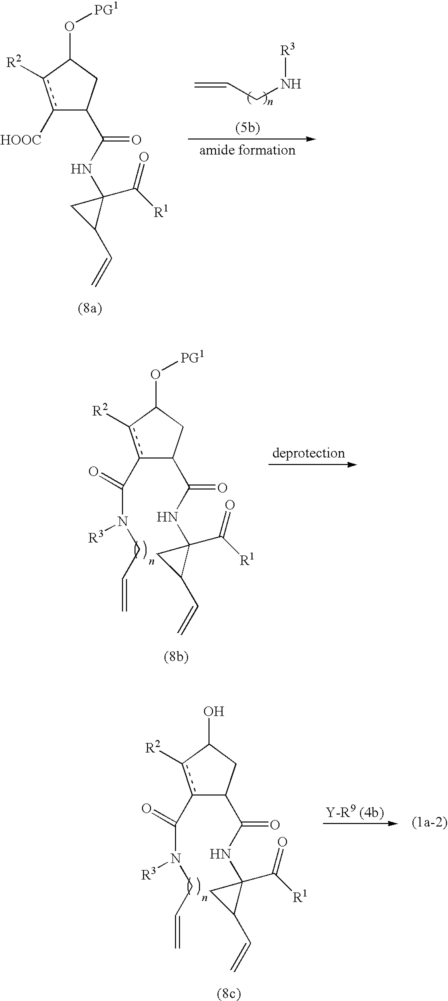

PG¹ is an O-protecting group as described above. The same reaction conditions as described above may be used: amide formation as described above, removal of PG¹ as in the description of the protecting groups and introduction of R⁹ as in the reactions of (4a) with the reagents (4b).

The intermediates of formula (2a) may be prepared by first cyclizing the open amide (9a) to a macrocycle ester (9b), which in turn is converted to (2a) as follows:

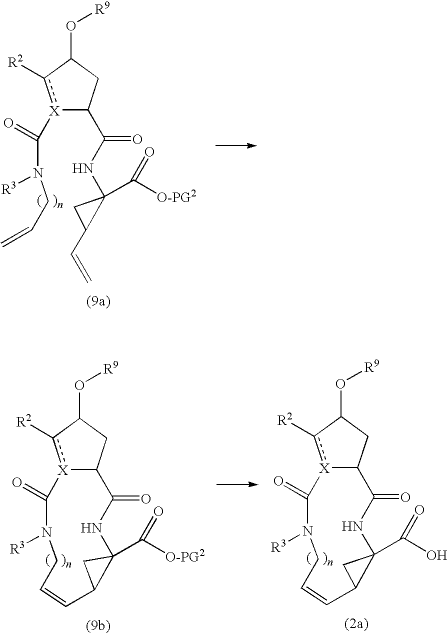

PG² is a carboxyl protecting group, e.g. one of the carboxyl protecting groups mentioned above, in particular a C₁₋₄ alkyl or benzyl ester, e.g. a methyl, ethyl or tert-butyl ester. The reaction of (9a) to (9b) is a metathesis reaction and is conducted as described above. The group PG² is removed following procedures also described above. Where PG¹ is a C₁₋₄ alkyl ester, it is removed by alkaline hydrolysis, e.g. with NaOH or preferably LiOH, in an aqueous solvent, e.g. a C₁₋₄alkanol/water mixture. A benzyl group can be removed by catalytic hydrogenation.

In an alternative synthesis, intermediates (2a) can be prepared as follows:

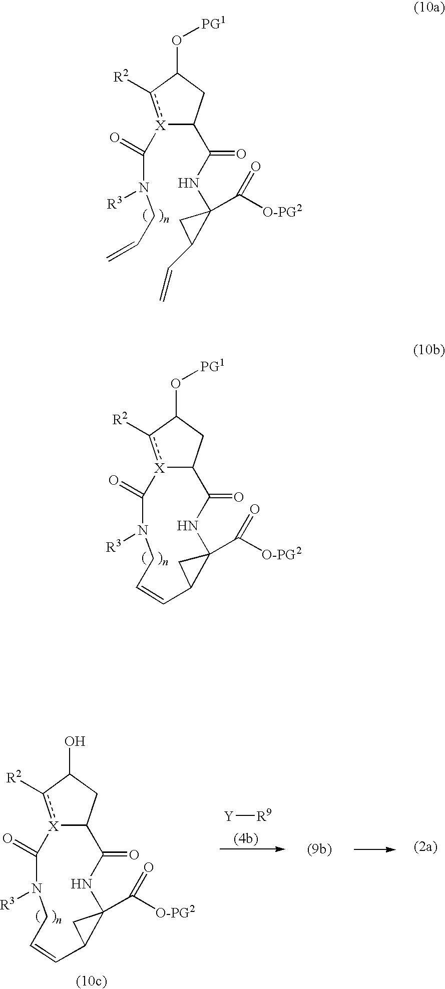

The PG¹ group is selected such that it is selectively cleavable towards PG². PG² may be e.g. methyl or ethyl esters, which can be removed by treatment with an alkali metal hydroxide in an aqueous medium, in which case PG¹ e.g. is tert-butyl or benzyl. PG² may be tert-butyl esters removable under weakly acidic conditions or PG¹ may be benzyl esters removable with strong acid or by catalytic hydrogenation, in the latter two cases PG¹ e.g. is a benzoic ester such as a 4-nitrobenzoic ester.

First, intermediates (10a) are cyclized to the macrocyclic esters (10b), the latter are deprotected by removal of the PG¹ group to (10c), which are reacted with intermediates (4b), followed by removal of carboxyl protecting group PG². The cyclization, deprotection of PG¹ and PG² and the coupling with (4b) are as described above.

The R¹ groups can be introduced at any stage of the synthesis, either as the last step as described above, or earlier, before the macrocycle formation. In the following scheme, the group R¹ being —NHSO₃R^{f} (which are as specified above) are introduced:

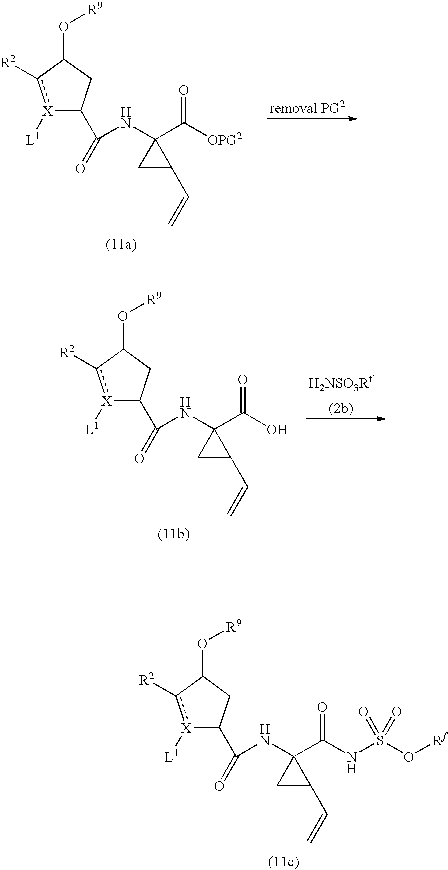

In the above scheme, PG² is as defined above and L¹ is a P3 group

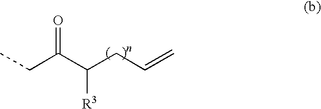

wherein n and R³ are as defined above and where X is N, L¹ may also be a nitrogen protecting group (PG, as defined above) and where X is C, L¹ may also be a group —COOPG^{2a}, wherein the group PG^{2a} is a carboxyl protecting group similar as PG², but wherein PG^{2a} is selectively cleavable towards PG². In one embodiment PG^{2a} is tert-butyl and PG² is methyl or ethyl.

The intermediates (11c) and (11d) wherein L¹ represents a group (b) correspond to the intermediates (1a) and may be processed further as specified above.

### Coupling of P1 and P2 Building Blocks

The P1 and P2 building blocks are linked using an amide forming reaction following the procedures described above. The P1 building block may have a carboxyl protecting group PG² (as in (12b)) or may already be linked to P1′ group (as in (12c)). L² is a N-protecting group (PG), or a group (b), as specified above. L³ is hydroxy, —OPG¹ or a group —O—R⁹ as specified above. Where in any of the following reaction schemes L³ is hydroxy, prior to each reaction step, it may be protected as a group —OPG¹ and, if desired, subsequently deprotected back to a free hydroxy function. Similarly as described above, the hydroxy function may be converted to a group —O—R⁹.

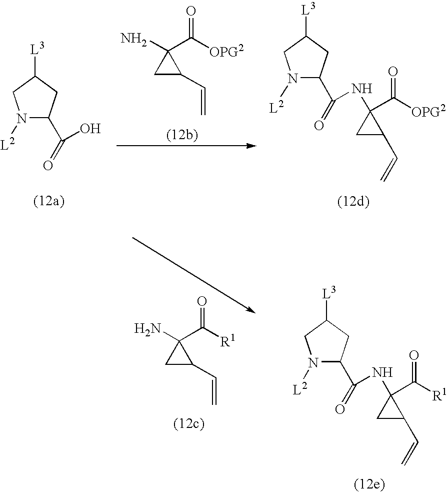

In the procedure of the above scheme, a cyclopropyl amino acid (12b) or (12c) is coupled to the acid function of the P2 building block (12a) with the formation of an amide linkage, following the procedures described above. Intermediates (12d) or (12e) are obtained. Where in the latter L² is a group (b), the resulting products are P3-P2-P1 sequences encompassing some of the intermediates (11c) or (11d) in the previous reaction scheme. Removal of the acid protecting group in (12d), using the appropriate conditions for the protecting group used, followed by coupling with an amine —NHSO₃R^{f} (2b) as described above, again yields the intermediates (12e), wherein —COR¹ is an amide group. Where L² is a N-protecting group, it can be removed yielding intermediates (5a) or (6a). In one embodiment, PG in this reaction is a BOC group and PG² is methyl or ethyl. Where additionally L³ is hydroxy, the starting material (12a) is Boc-L-hydroxyproline. In a particular embodiment, PG is BOC, PG² is methyl or ethyl and L³ is —O—R⁹.

In one embodiment, L² is a group (b) and these reactions involve coupling P1 to P2-P3, which results in the intermediates (1a-1) or (1a) mentioned above. In another embodiment, L² is a N-protecting group PG, which is as specified above, and the coupling reaction results in intermediates (12d-1) or (12e-1), from which the group PG can be removed, using reaction conditions mentioned above, obtaining intermediates (12-f) or respectively (12g), which encompass intermediates (5a) and (6a) as specified above:

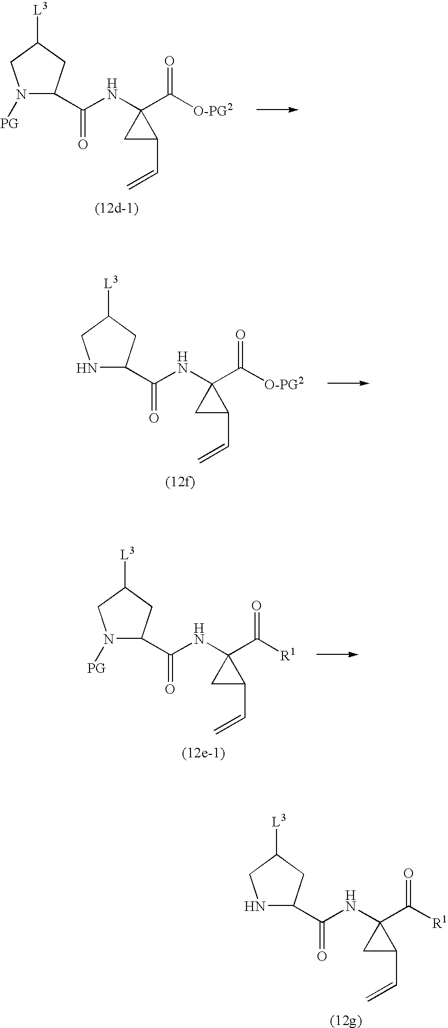

In one embodiment, the group L³ in the above schemes represents a group —O-PG¹ which can be introduced on a starting material (12a) wherein L³ is hydroxy. In this instance PG¹ is chosen such that it is selectively cleavable towards group L² being PG.

In a similar way, P2 building blocks wherein X is C, which are cyclopentane or cyclopentene derivatives, can be linked to PI building blocks as outlined in the following scheme wherein R¹, R², L³ are as specified above and PG² and PG^{2a} are carboxyl protecting groups. PG^{2a} typically is chosen such that it is selectively cleavable towards group PG². Removal of the PG^{2a} group in (13c) yields intermediates (7a) or (8a), which can be reacted with (5b) as described above.

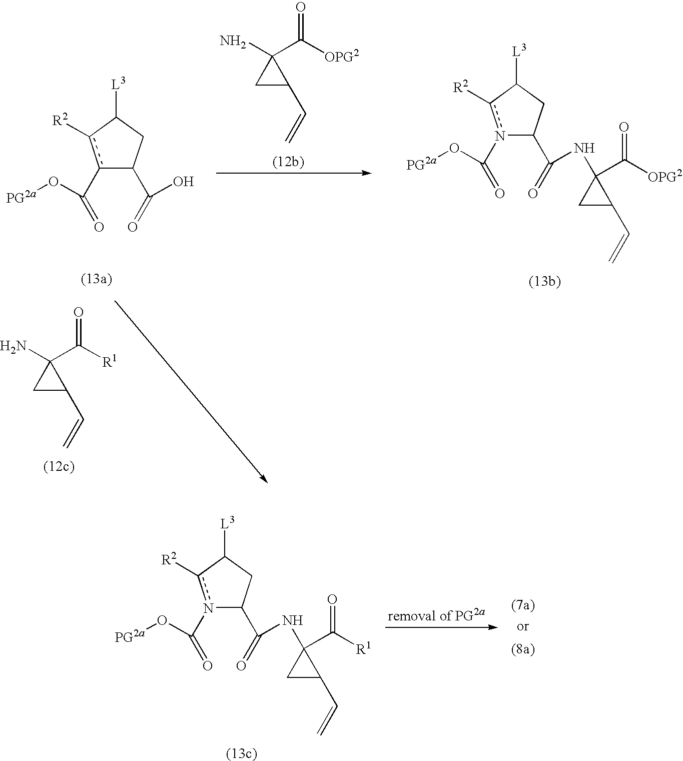

In a particular embodiment, where X is C, R² is H, and where X and the carbon bearing R² are linked by a single bond (P2 being a cyclopentane moiety), PG^{2a} and L³ taken together form a bond and the P2 building block is represented by formula:

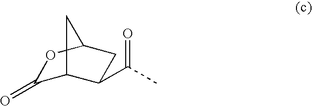

Bicyclic acid (14a) is reacted with (12b) or (12c) similar as described above to (14b) and (14c) respectively, wherein the lactone is opened giving intermediates (14c) and (14e). The lactones can be opened using ester hydrolysis procedures, for example using the reaction conditions described above for the alkaline removal of a PG¹ group in (9b), in particular using basic conditions such as an alkali metal hydroxide, e.g. NaOH, KOH, in particular LiOH.

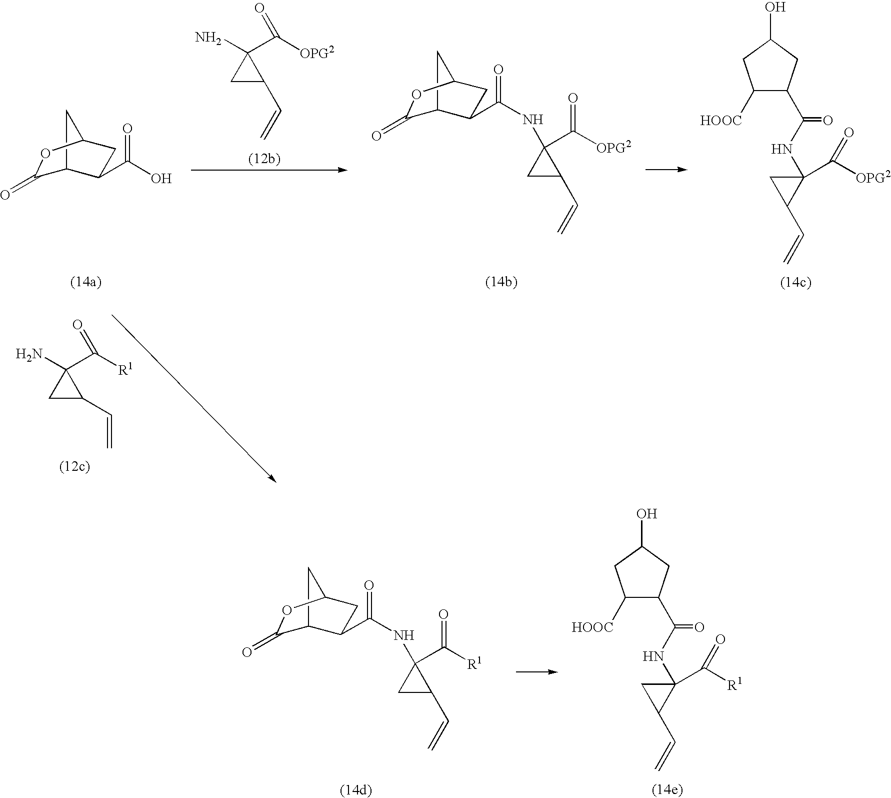

Intermediates (14c) and (14e) can be processed further as described hereinafter.

### Coupling of P3 and P2 Building Blocks

For P2 building blocks that have a pyrrolidine moiety, the P3 and P2 or P3 and P2-P1 building blocks are linked using a carbamate forming reaction following the procedures described above for the coupling of (5a) with (5b). A general procedure for coupling P2 blocks having a pyrrolidine moiety is represented in the following reaction scheme wherein L³ is as specified above and L⁴ is a group —O-PG², a group

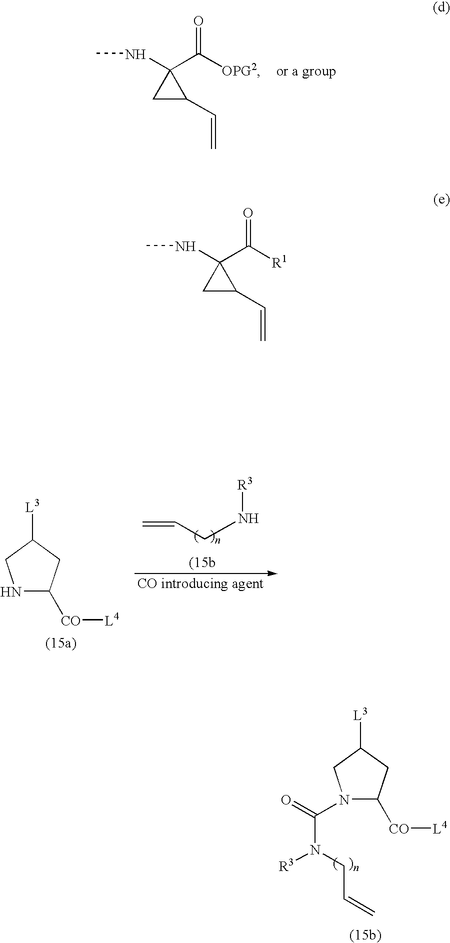

In one embodiment L⁴ in (15a) is a group —OPG², the PG² group may be removed and the resulting acid coupled with cyclopropyl amino acids (12a) or (12b), yielding intermediates (12d) or (12e) wherein L² is a radical (d) or (e).

A general procedure for coupling P3 blocks with a P2 block or a with a P2-P1 block wherein the P2 is a cyclopentane or cyclopentene is shown in the following scheme. L³ and L⁴ are as specified above.

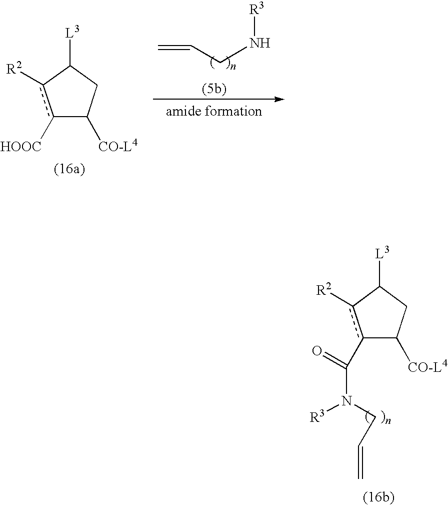

In a particular embodiment L³ and L⁴ taken together may form a lactone bridge as in (14a), and the coupling of a P3 block with a P2 block is as follows:

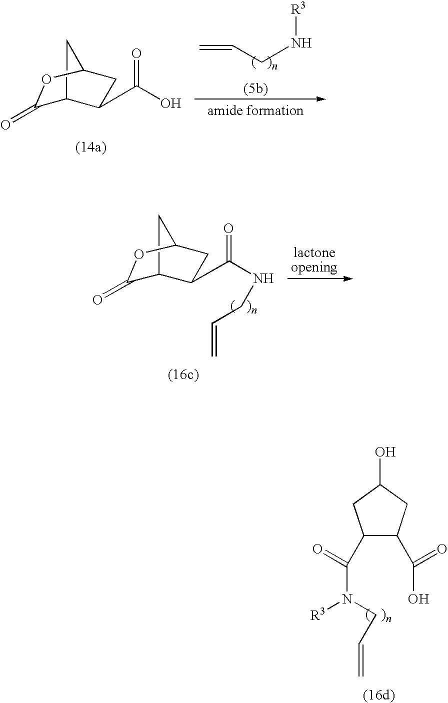

Bicyclic lactone (14a) is reacted with (5b) in an amide forming reaction to amide (16c) in which the lactone bridge is opened to (16d). The reaction conditions for the amide forming and lactone opening reactions are as described above or hereinafter. Intermediate (16d) in turn can be coupled to a P1 group as described above.

The reactions in the above schemes are conducted using the same procedures as described above for the reactions of (5a), (7a) or (8a) with (5b) and in particular the 1 above reactions wherein L⁴ is a group (d) or (e) correspond to the reactions of (5a), (7a) or (8a) with (5b), as described above.

The building blocks P1, P1′, P2 and P3 used in the preparation of the compounds of formula (I) can be prepared starting from art-known intermediates. A number of such syntheses are described hereafter in more detail.

The individual building blocks can first be prepared and subsequently coupled together or alternatively, precursors of the building blocks can be coupled together and modified at a later stage to the desired molecular composition.

The functionalities in each of the building blocks may be protected to avoid side reactions.

### Synthesis of P2 Building Blocks

The P2 building blocks contain either a pyrrolidine, a cyclopentane, or a cyclopentane moiety substituted with a group —O—R⁴.

P2 building blocks containing a pyrrolidine moiety can be derived from commercially available hydroxyproline.

The preparation of P2 building blocks that contain a cylopentane ring may be perfouned as shown in the scheme below.

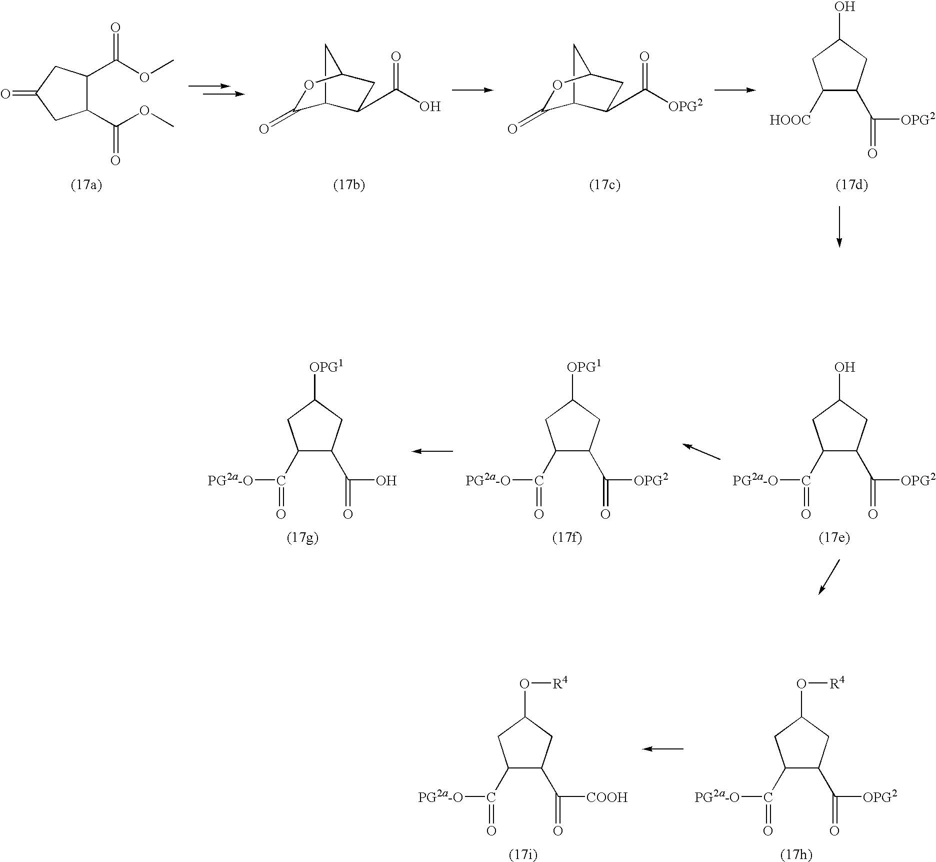

The bicyclic acid (17b) can be prepared, for example, from 3,4-bis(methoxycarbonyl)-cyclopentanone (17a), as described by Rosenquist et al. in Acta Chem. Scand. 46 (1992) 1127-1129. A first step in this procedure involves the reduction of the keto group with a reducing agent like sodium borohydride in a solvent such as methanol, followed by hydrolysis of the esters and finally ring closure to the bicyclic lactone (17b) using lactone forming procedures, in particular by using acetic anhydride in the presence of a weak base such as pyridine. The carboxylic acid functionality in (17b) can then be protected by introducing an appropriate carboxyl protecting group, such as a group PG², which is as specified above, thus providing bicyclic ester (17c). The group PG² in particular is acid-labile such as a tert-butyl group and is introduced e.g. by treatment with isobutene in the presence of a Lewis acid or with di-tert-butyl dicarbonate in the presence of a base such as a tertiary amine like dimethylaminopyridine or triethylamine in a solvent like dichloromethane. Lactone opening of (17c) using reaction conditions described above, in particular with lithium hydroxide, yields the acid (17d), which can be used further in coupling reactions with P1 building blocks. The free acid in (17d) may also be protected, preferably with an acid protecting group PG^{2a} that is selectively cleavable towards PG², and the hydroxy function may be converted to a group —OPG¹ or to a group —O—R⁹. The products obtained upon removal of the group PG² are intermediates (17g) and (17i) which correspond to intermediates (13a) or (16a) specified above.

Intermediates with specific stereochemistry may be prepared by resolving the intermediates in the above reaction sequence. For example, (17b) may be resolved following art-known procedures, e.g. by salt form action with an optically active base or by chiral chromatography, and the resulting stereoisomers may be processed further as described above. The OH and COOH groups in (17d) are in cis position. Trans analogs can be prepared by inverting the stereochemistry at the carbon bearing the OH function by using specific reagents in the reactions introducing OPG¹ or O—R⁹ that invert the stereochemistry, such as, e.g. by applying a Mitsunobu reaction.

In one embodiment, the intermediates (17d) are coupled to P1 blocks (12b) or (12c), which coupling reactions correspond to the coupling of (13a) or (16a) with the same P1 blocks, using the same conditions. Subsequent introduction of a —O—R⁹ substituent as described above followed by removal of the acid protection group PG² yields intermediates (8a-1), which are a subclass of the intermediates (7a), or part of the intermediates (16a). The reaction products of the PG² removal can be further coupled to a P3 building block In one embodiment PG² in (17d) is tort-butyl which can be removed under acidic conditions, e.g. with trifluoroacetic acid.

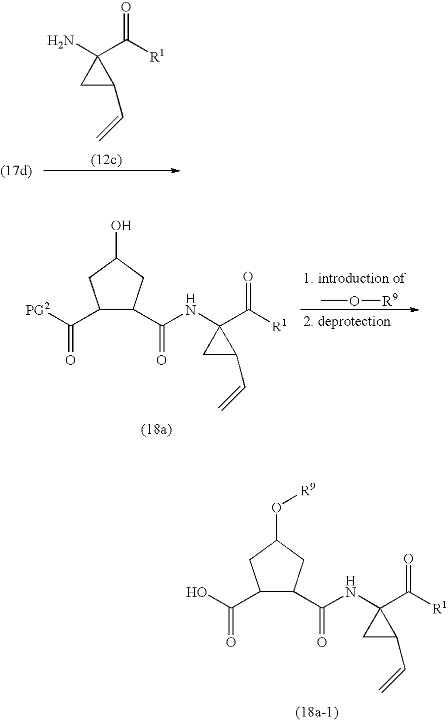

An unsaturated P2 building block, i.e. a cyclopentene ring, may be prepared as illustrated in the scheme below.

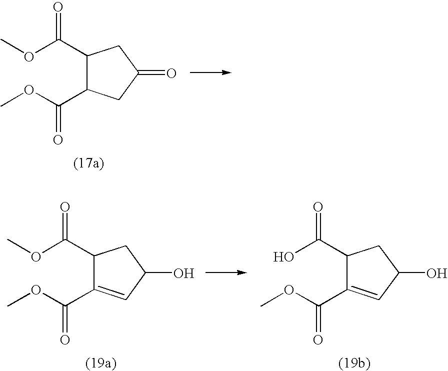

A bromination-elimination reaction of 3,4-bis(methoxycarbonyl)cyclopentanone (17a) as described by Dolby et al. in J. Org. Chem. 36 (1971) 1277-1285 followed by reduction of the keto functionality with a reducing agent like sodium borohydride provides the cyclopentenol (19a). Selective ester hydrolysis using for example lithium hydroxide in a solvent like a mixture of dioxane and water, provides the hydroxy substituted monoester cyclopentenol (19b).

An unsaturated P2 building block wherein R² can also be other than hydrogen, may be prepared as shown in the scheme below.

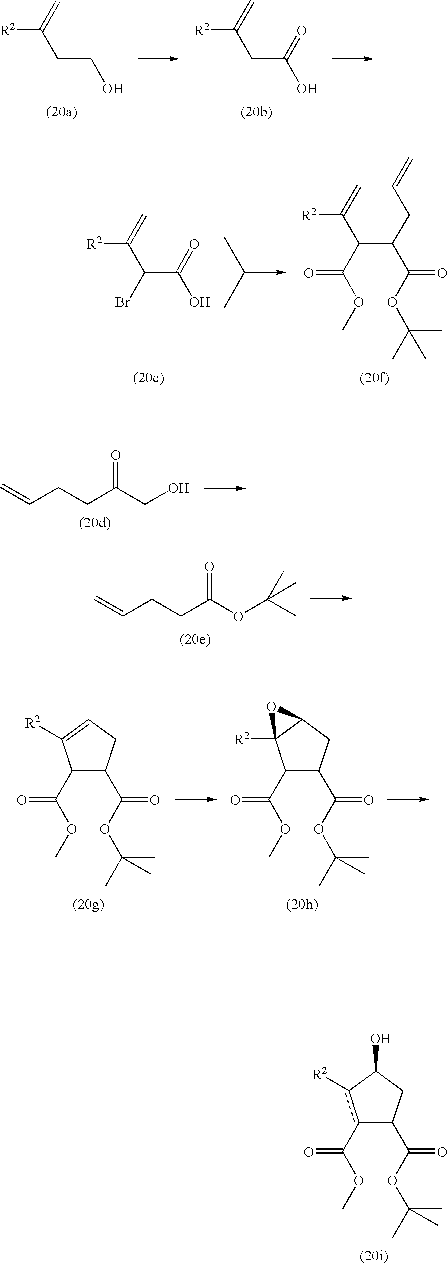

Oxidation of commercially available 3-methyl-3-buten-1-ol (20a), in particular by an oxidizing agent like pyridinium chlorochromate, yields (20b), which is converted to the corresponding methyl ester, e.g. by treatment with acetyl chloride in methanol, followed by a bromination reaction with bromine yielding the a-bromo ester (20c). The latter can then be condensed with the alkenyl ester (20e), obtained from (20d) by an ester forming reaction. The ester in (20e) preferably is a tert-butyl ester which can be prepared from the corresponding commercially available acid (20d), e.g. by treatment with di-tert-butyl Bicarbonate in the presence of a base like dimethylaminopyridine.

Intermediate (20e) is treated with a base such as lithium diisopropyl amide in a solvent like tetrahydrofuran, and reacted with (20c) to give the alkenyl diester (20f). Cyclisation of (20f) by an olefin metathesis reaction, performed as described above, provides cyclopentene derivative (20g). Stereoselective epoxidation of (20g) can be carried out using the Jacobsen asymmetric epoxidation method to obtain epoxide (20h). Finally, an epoxide opening reaction under basic conditions, e.g. by addition of a base, in particular DBN (1,5-diazabicyclo-[4.3.0]non-5-ene), yields the alcohol (20i). Optionally, the double bond in intermediate (20i) can be reduced, for example by catalytic hydrogenation using a catalyst like palladium on carbon, yielding the corresponding cyclopentane compound. The tert-butyl ester may be removed to the corresponding acid, which subsequently is coupled to a P1 building block.

The —R⁹ group can be introduced on the pyrrolidine, cyclopentane or cyclopentene rings at any convenient stage of the synthesis of the compounds according to the present invention. One approach is to first introduce the —R⁹ group to the said rings and subsequently add the other desired building blocks, i.e. P1 (optionally with the P1′ tail) and P3, followed by the macrocycle formation. Another approach is to couple the building blocks P2, bearing no —O—R⁹ substituent, with each P1 and P3, and to add the —R⁹ group either before or after the macrocycle formation. In the latter procedure, the P2 moieties have a hydroxy group, which may be protected by a hydroxy protecting group PG¹.

R⁹ groups can be introduced on building blocks P2 by reacting hydroxy substituted intermediates (21a) or (21b) with intermediates (4b) similar as described above for the synthesis of (I) starting from (4a). These reactions are represented in the schemes below, wherein L² is as specified above and L⁵ and L5^{a} independently from one another, represent hydroxy, a carboxyl protecting group —OPG² or —PG^{2a}, or L⁵ may also represent a P1 group such as a group (d) or (e) as specified above, or L⁵a may also represent a P3 group such as a group (b) as specified above The groups PG² and PG^{2a} are as specified above. Where the groups L⁵ and L^{5a} are PG² or PG^{2a}, they are chosen such that each group is selectively cleavable towards the other. For example, one of L⁵ and L^{5a} may be a methyl or ethyl group and the other a benzyl or tert-butyl group.

In one embodiment in (21a) L² is PG and L⁵ is —OPG², or in (21d), L^{5a} is —OPG² and L⁵ is —OPG² and the PG² groups are removed as described above.

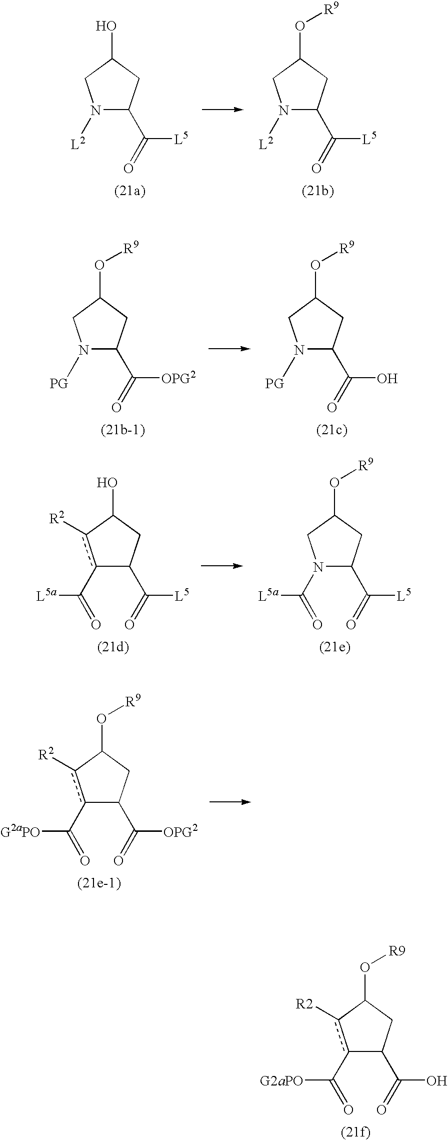

Alternatively, when handling hydroxy substituted cyclopentane analogues, the quinoline substituent can be introduced via a similar Mitsunobu reaction by reacting the hydroxy group of compound (2a′) with the desired alcohol (3b) in the presence of triphenylphosphine and an activating agent like diethyl azodicarboxylate (DEAD), diisopropyl azodicarboxylate (DIAD) or the like.

In another embodiment the group L² is Boc, L⁵ is hydroxy and the starting material (21a) is commercially available BOC-hydroxyproline, or any other stereoisomeric form thereof, e.g. Boc-L-hydroxyproline, in particular the trans isomer of the latter. Where L⁵ in (21b) is a carboxyl-protecting group, it may be removed following procedures described above to (21c). In still another embodiment PG in (21b-1) is Boc and PG² is a lower alkyl ester, in particular a methyl or ethyl ester. Hydrolysis of the latter ester to the acid can be done by standard procedures, e.g. acid hydrolysis with hydrochloric acid in methanol or with an alkali metal hydroxide such as NaOH, in particular with LiOH. In another embodiment, hydroxy substituted cyclopentane or cyclopentene analogs (21d) are converted to (21e), which, where L⁵ and L⁵a are —OPG² or —OPG^{2a}, may be converted to the corresponding acids (21f) by removal of the group PG². Removal of PG^{2a} in (21e-1) leads to similar intermediates.

The intermediates Y—R⁹ (4b) can be prepared following art-known methods using known starting materials. A number of synthesis pathways for such intermediates will be described hereafter in somewhat more detail. For example the preparation of the above mentioned intermediate quinolines is shown below in the following scheme.

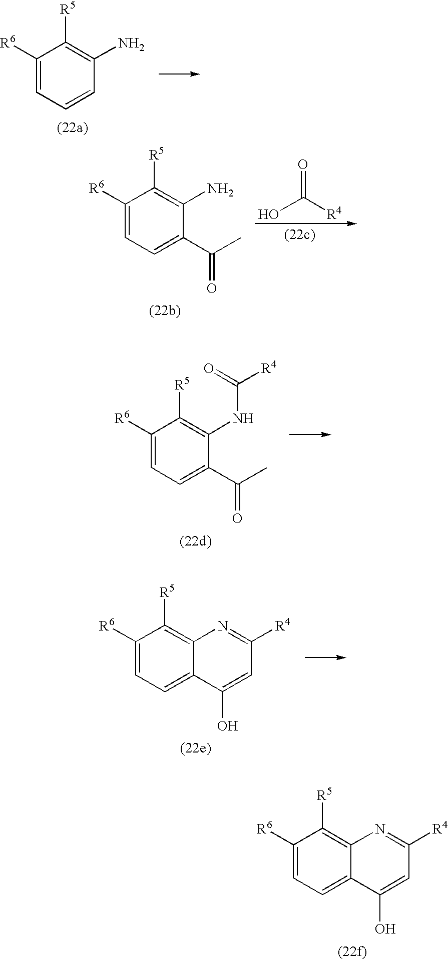

Friedel-Craft acylation of a suitable substituted aniline (22a), available either commercially or via art-known procedures, using an acylating agent such as acetyl chloride or the like in the presence of one or more Lewis acid such as boron trichloride and aluminum trichloride in a solvent like dichloromethane provides (22b). Coupling of (22b) with a carboxylic acid (22c), preferably under basic conditions, such as in pyridine, in the presence of an activating agent for the carboxylate group, for instance POCl₃, followed by ring closure and dehydration under basic conditions like potassium tert-butoxide in tert-butanol yields quinoline derivative (22e). The latter can be converted to (22f) wherein LG is a leaving group, e.g. by reaction of (22e) with a halogenating agent, for example phosphoryl chloride or the like, or with an arylsulfonyl chloride, e.g. with tosyl chloride. Quinoline derivative (22e) can be coupled in a Mitsunobu reaction to an alcohol as described above, or quinoline (22f) can be reacted with (1a) in an O-arylation reaction as described above.

A variety of carboxylic acids with the general structure (22c) can be used in the above synthesis. These acids are available either commercially or can be prepared via art-known procedures. An example of the preparation of 2-carboxy-4-(substituted)thiazole (22c-1), following the procedure described by Berdikhina et. al. in Chem. Heterocycl. Compd. (Engl. Transl.) (1991), 427-433, is shown in the following reaction scheme which illustrates the preparation of 2-carboxy-4-isopropylthiazole (22c-1):

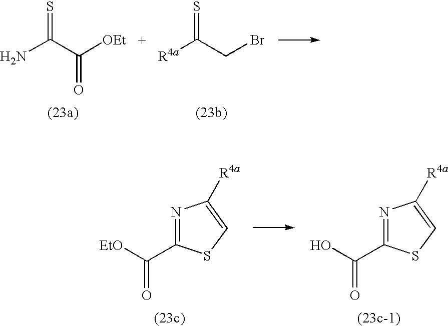

Ethyl thiooxamate (23a) is reacted with the a-bromoketone (23b) to form the ethyl thiazolyl carboxylic acid ester (23c), which is hydrolyzed to the corresponding acid (22c-1). The ethyl ester in these intermediates may be replaced by other carboxyl protecting groups PG², as defined above. In the above scheme R^{4a} is as defined above and in particular is C₁₋₄ alkyl, more in particular isopropyl.

The a-bromoketone (23b) may be prepared from 3-methyl-butan-2-one (MIK) with a sililating agent (such as TMSCl) in the presence of a suitable base (in particular LiHMDS) and bromine.

The synthesis of further carboxylic acids (22c), in particular of substituted amino thiazole carboxylic acids (22c-2) is illustrated hereinbelow:

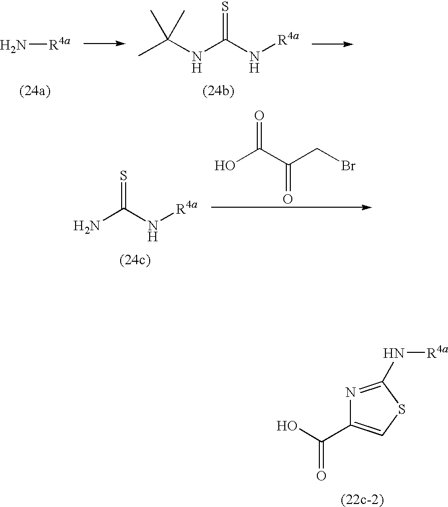

Thiourea (24c) with various substituents R^{4a}, which in particular are C₁₋₆alkyl, can be formed by reaction of the appropriate amine (24a) with tert-butylisothiocyanate in the presence of a base like diisopropylethylamine in a solvent like dichloromethane followed by removal of the tert-butyl group under acidic conditions. Subsequent condensation of thiourea derivative (24c) with 3-bromopyruvic acid provides the thiazole carboxylic acid (22c-2).

### Synthesis of P1 Building Blocks

The cyclopropane amino acid used in the preparation of the P1 fragment is commercially available or can be prepared using art-known procedures.

In particular the amino-vinyl-cyclopropyl ethyl ester (12b) may be obtained according to the procedure described in WO 00/09543 or as illustrated in the following scheme, wherein PG² is a carboxyl protecting group as specified above:

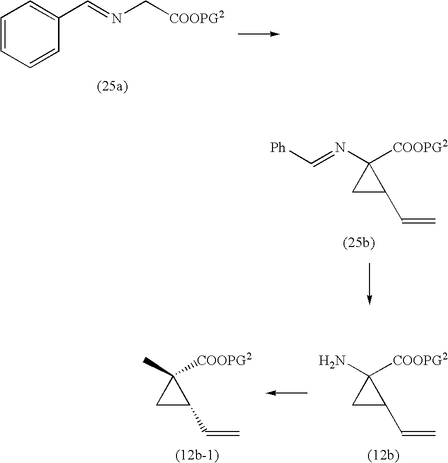

Treatment of commercially available or easily obtainable imine (25a) with 1,4-dihalobutene in presence of a base produces (25b), which after hydrolysis yields cyclopropyl amino acid (12b), having the allyl substituent syn to the carboxyl group. Resolution of the enantiomeric mixture (12b) results in (12b-1). The resolution is performed using art-known procedures such as enzymatic separation; crystallization with a chiral acid; or chemical derivatization; or by chiral column chromatography. Intermediates (12b) or (12b-1) may be coupled to the appropriate P2 derivatives as described above.

P1 building blocks for the preparation of compounds according to general formula (I) wherein R¹ is —NHSO₃R^{f} can be prepared by reacting amino acids (26a) with the appropriate amine under standard conditions for amide formation. Cyclopropyl amino acids (26a) are prepared by introducing a N-protecting group PG, and removal of PG² and the resulting amino acids (26a) are converted to the amides (12c-1), which are subgroups of the intermediates (12c), as outlined in the following reaction scheme, wherein PG is as specified above.

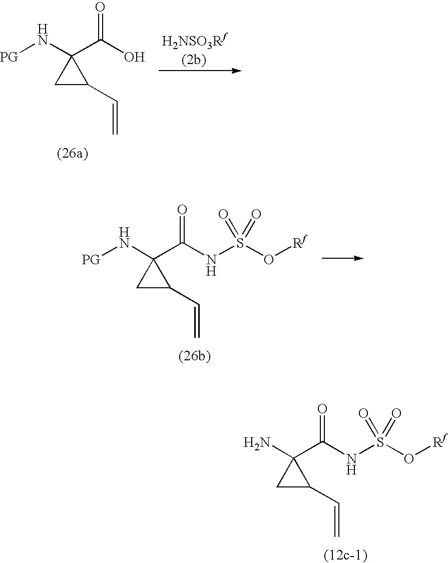

The reaction of (26a) with amine (2b) is an amide forming procedure and can be performed following the procedures described above. This reaction yields intermediates (26b) from which the amino protecting group is removed by standard methods such as those described above. This in turn results in the desired intermediate (12c-1). Starting materials (26a) may be prepared from the above-mentioned intermediates (12b) by first introducing a N-protecting group PG and subsequent removal of the group PG².

In one embodiment the reaction of (26a) with (2b) is done by treatment of the amino acid with a coupling agent, for example 0-(7-azabenzotriazol-1-yl)-N,N,N,′,N′-tetramethyluronium hexafluorophosphate (HATU) in the presence of diisopropylethylamine, in a solvent such as dichloromethane or DMF followed by reaction with (2b) in the presence of a base such as 1,8-diazabicyclo[4.0]undec-7-ene (DBU). Alternatively, N,N′-carbonyldiimidazole (CDI) or the like, in a solvent like THF in the presence of a base such as DBU can also be used in the coupling of (26a) with (2b). Another alternative procedure involves the amino acid (26a) being treated with (2b) in the presence of a base like diisopropylethylamine followed by treatment with a coupling agent such as benzotriazole-1-yl-oxy-tris-pyrrolidinophosphonium hexafluorophosphate (commercially available as PyBOP®), to effect the introduction of the sulfamate group.

Intermediate (12c-1) in turn may be coupled to the appropriate proline, cyclopentane or cyclopentene derivatives as described above.

### Synthesis of the P3 Building Blocks

The P3 building blocks are available commercially or can be prepared according to methodologies known to the skilled in the art. One of these methodologies is shown in the scheme below and uses monoacylated amines, such as a trifluoroacetamide or a Boc protected amine.

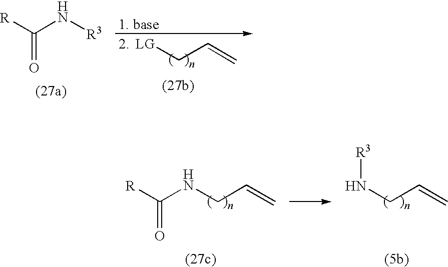

In the above scheme, R together with the CO group forms a N-protecting group, in particular R is Pert-butoxy or trifluoromethyl; R³ and n are as defined above and LG is a leaving group, in particular halogen, e.g. chloro or bromo.

The monoacylated amines (27a) are treated with a strong base such as sodium hydride and are subsequently reacted with a reagent LG-C₅₋₈alkenyl (27b), in particular haloC₅₋₈alkenyl, to form the corresponding protected amines (27c). Deprotection of (27e) affords (5b), which are building blocks P3. Deprotection will depend on the functional group R, thus if R is tert-butoxy, deprotection of the corresponding Sac-protected amine can be accomplished with an acidic treatment, e.g. trifluoroacetic acid. Alternatively, when R is for instance trifluoromethyl, removal of the R group is accomplished with a base, e.g. sodium hydroxide.

The following scheme illustrates yet another method for preparing a P3 building block, namely a Gabriel synthesis of primary C₅₋₈alkenylamines, which can be carried out by the treatment of a phthalimide (28a) with a base, such as NaOH or KOH, and with (27b), which is as specified above, followed by deprotection of the intermediate N-alkenyl imide with a reagent such as hydrazine monohydrate to generate a primary C₅₋₈alkenylamine (5b-1).

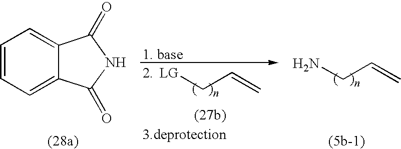

In the above scheme, n is as defined above.

### Synthesis of P1′ Building Blocks

P1′ building blocks can be prepared according to methodologies known to the skilled in the art from commercially available starting materials via a two step process. First, chlorosulfonyl isocyanate (29a) is reduced with a suitable reagent to chlorosulfonyl amide (29b). The chlorosulfonyl amide (29b) can then undergo esterification with an appropriate alcohol R^{f}OH (29c) in a suitable organic solvent such as NMP to form the corresponding sulfamate (2b), which can be readily isolated by crystallization or chromatography.

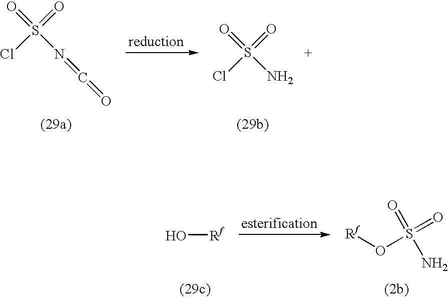

In one embodiment, by way of example and not limitation, the conversion of (29a) to (29b) is accomplished by treating (29a) with formic acid to afford reduction to chlorosulfonyl amide (29b), which is then subsequently treated with R^{f}OH to afford sulfamate (2b). R^{f}OH is an alcohol as defined above and in particular R^{f} is C₃₋₅cycloalkyl, more in particular cyclopropanol or 1-methyl-1-cyclopropanol which can be prepared according to methodologies known to the skilled in the art with reference to procedures and intermediates described by Krow, G R., et al. Organic Reactions, 1993, 43; Denis or J. M. et. al. Synthesis, 1972, 10, 549 and Kulinkovich, O. G., et. al. Synthesis, 1991, 3, 234.

Compounds of formula (I) may be converted into each other following art-known functional group transformation reactions. For example, amino groups may be N-alkylated, nitro groups reduced to amino groups, a halo atom may be exchanged for another halo.

The compounds of formula (I) or (II) may be converted to the corresponding N-oxide forms following art-known procedures for converting a trivalent nitrogen into its N-oxide form. Said N-oxidation reaction may generally be carried out by reacting the starting material of formula (I) or (II) with an appropriate organic or inorganic peroxide. Appropriate inorganic peroxides comprise, for example, hydrogen peroxide, alkali metal or earth alkaline metal peroxides, e.g. sodium peroxide, potassium peroxide, appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Pure stereochemically isomeric forms of the compounds of formula (I) or (II) may be obtained by the application of art-known procedures. Diastereomers may be separated by physical methods such as selective crystallization and chromatographic techniques, e.g., counter-current distribution, liquid chromatography and the like.

The compounds of formula (I) or (II) may be obtained as racemic mixtures of enantiomers which can be separated from one another following art-known resolution procedures. The racemic compounds of formula (I) or (II), which are sufficiently basic or acidic may be converted into the corresponding diastereomeric salt forms by reaction with a suitable chiral acid, respectively chiral base. Said diastereomeric salt forms are subsequently separated, for example, by selective or fractional crystallization and the enantiomers are liberated therefrom by alkali or acid. An alternative manner of separating the enantiomeric forms of the compounds of formula (I) or (II) involves liquid chromatography, in particular liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound may be synthesized by stereospecific methods of preparation. These methods may advantageously employ enantiomerically pure starting materials.

In a further aspect, the present invention concerns a pharmaceutical composition comprising a therapeutically effective amount of a compound of formula (I) or (II) as specified herein, or a compound of any of the subgroups of compounds of formula (I) or (II) as specified herein, and a pharmaceutically acceptable carrier. A therapeutically effective amount in this context is an amount sufficient to prophylactically act against, to stabilize or to reduce viral infection, and in particular HCV viral infection, in infected subjects or subjects being at risk of being infected. In still a further aspect, this invention relates to a process of preparing a pharmaceutical composition as specified herein, which comprises intimately mixing a pharmaceutically acceptable carrier with a therapeutically effective amount of a compound of formula (I) or (II), as specified herein, or of a compound of any of the subgroups of compounds of formula (I) or (II) as specified herein.

Therefore, the compounds of the present invention or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form or metal complex, as the active ingredient is combined in intimate admixture with a pharmaceutically acceptable carrier, which carrier may take a wide variety of forms depending on the fouli of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

The compounds of the present invention may also be administered via oral inhalation or insufflation by means of methods and formulations employed in the art for administration via this way. Thus, in general the compounds of the present invention may be administered to the lungs in the form of a solution, a suspension or a dry powder, a solution being preferred. Any system developed for the delivery of solutions, suspensions or dry powders via oral inhalation or insufflation are suitable for the administration of the present compounds.

Thus, the present invention also provides a pharmaceutical composition adapted for administration by inhalation or insufflation through the mouth comprising a compound of formula (I) or (II) and a pharmaceutically acceptable carrier. Preferably, the compounds of the present invention are administered via inhalation of a solution in nebulized or aerosolized doses.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The compounds of formula (I) or (II) show antiviral properties. Viral infections and their associated diseases treatable using the compounds and methods of the present invention include those infections brought on by HCV and other pathogenic flaviviruses such as Yellow fever, Dengue fever (types 1-4), St. Louis encephalitis, Japanese encephalitis, Murray valley encephalitis, West Nile virus and Kunjin virus. The diseases associated with HCV include progressive liver fibrosis, inflammation and necrosis leading to cirrhosis, end-stage liver disease, and HCC; and for the other pathogenic flaviviruses the diseases include yellow fever, dengue fever, hemorrhagic fever and encephalitis. A number of the compounds of this invention moreover are active against mutated strains of HCV. Additionally, many of the compounds of this invention show a favorable pharmacokinetic profile and have attractive properties in terms of bioavailabilty, including an acceptable half-life, AUC (area under the curve) and peak values and lacking unfavorable phenomena such as insufficient quick onset and tissue retention.

Due to their antiviral properties, particularly their anti-HCV properties, the compounds of formula (I) or (II) or any subgroup thereof, their prodrugs, N-oxides, addition salts, quaternary amines, metal complexes and stereochemically isomeric forms, are useful in the treatment of individuals experiencing a viral infection, particularly a HCV infection, and for the prophylaxis of these infections. In general, the compounds of the present invention may be useful in the treatment of warm-blooded animals infected with viruses, in particular flaviviruses such as HCV.

The compounds of the present invention or any subgroup thereof may therefore be used as medicines. Said use as a medicine or method of treatment comprises the systemic administration to viral infected subjects or to subjects susceptible to viral infections of an amount effective to combat the conditions associated with the viral infection, in particular the HCV infection.

The present invention also relates to the use of the present compounds or any subgroup thereof in the manufacture of a medicament for the treatment or the prevention of viral infections, particularly HCV infection.

The present invention furthermore relates to a method of treating a warm-blooded animal infected by a virus, or being at risk of infection by a virus, in particular by HCV, said method comprising the administration of an anti-virally effective amount of a compound of formula (I) or (II), as specified herein, or of a compound of any of the subgroups of compounds of formula (I) or (II), as specified herein.

### Combination Therapy

Combinations of one or more compounds of the present invention and one or more additional pharmaceutically active agent(s) may be used in the practice of the present invention to treat human beings having an HCV infection. Useful active therapeutic agents for treating an HCV infection include interferons, ribavirin or its analogs, HCV NS3 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophillin inhibitors, HCV IRES inhibitors, and pharmacokinetic enhancers.

More specifically, other active therapeutic ingredients or agents for treating HCV include:

(1) interferons selected from the group consisting of pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha-2b XL, BLX-883 (Locteron), DA-3021, glycosylated interferon alpha-2b (AVI-005), PEG-Infergen, PEGylated interferon lambda-1 (PEGylated TL-29), belerofon, and mixtures thereof;
(2) ribavirin and its analogs selected from the group consisting of ribavirin (Rebetol, Copegus), taribavirin (Viramidine), and mixtures thereof;
(3) HCV NS3 protease inhibitors selected from the group consisting of boceprevir (SCH-503034, SCH-7), telaprevir (VX-950), TMC-435350, BI-1335, BI-1230, MK-7009, VBY-376, VX-500, BMS-790052, BMS-605339, PHX-1766, AS-101, YH-5258, YH5530, YH5531, ITMN-191, and mixtures thereof;
(4) alpha-glucosidase 1 inhibitors selected from the group consisting of celgosivir (MX-3253), Miglitol, UT-231B, and mixtures thereof;
(5) hepatoprotectants selected from the group consisting of IDN-6556, ME 3738, LB-84451, silibilin, MitoQ, and mixtures thereof;
(6) nucleoside or nucleotide inhibitors of HCV NS5B polymerase selected from the group consisting of R1626, R7128 (R4048), 1DX184, IDX-102, BCX-4678, valopicitabine (NM-283), MK-0608, and mixtures thereof;
(7) non-nucleoside inhibitors of HCV NS5B polymerase selected from the group consisting of PF-868554, VCH-759, VCH-916, ITK-652, MK-3281, VBY-708, VCH-222, A848837, ANA-598, GL60667, GL59728, A-63890, A-48773, A-48547, BC-2329, VCH-796 (nesbuvir), GSK625433, BILN-1941, XTL-2125, GS-9190, and mixtures thereof;
(8) HCV NS5A inhibitors selected from the group consisting of AZD-2836 (A-831), A-689, and mixtures thereof;
(9) TLR-7 agonists selected from the group consisting of ANA-975, SM-360320, and mixtures thereof;
(10) cyclophillin inhibitors selected from the group consisting of DEBIO-025, SCY-635, NIM811, and mixtures thereof;
(11) HCV IRES inhibitors selected from the group consisting of MCI-067,
(12) pharmacokinetic enhancers selected from the group consisting of BAS-100, SPI-452, PF-419-4477, TMC-41629, roxythromycin, and mixtures thereof; and
(13) other drugs for treating HCV selected from the group consisting of thymosin alpha 1 (Zadaxin), nitazoxanide (Alinea, NTZ), BIVN-401 (virostat), PYN-17 (altirex), KPE02003002, actilon (CPG-10101), KRN-7000, civacir, GI-5005, XTL-6865, BIT225, PTX-111, ITX2865, TT-033i, ANA 971, NOV-205, tarvacin, EHC-18, VGX-410C, EMZ-702, AVT 4065, BMS-650032, BMS-791325, Bavituximab, MDX-1106 (ONO-4538), Oglufanide, VX-497 (merimepodib), and mixtures thereof.

Thus, in a further embodiment, the present invention provides a combination pharmaceutical composition comprising:

a) a compound of the present invention or a pharmaceutically acceptable salt thereof; and b) a second pharmaceutically active agent (or pharmaceutically acceptable salt thereof) effective to treat HCV.

In yet another embodiment, the present application provides a method for treating an HCV infection, wherein the method comprises the step of co-administering, to a human being in need thereof, a therapeutically effective amount of a compound of the present invention and one or more of the additional active agents described herein that are effective to treat HCV.

In the practice of this aspect of the invention, typically the amounts of a compound of the present invention and the one or more additional therapeutic agent(s) are individually therapeutic, but it is within the scope of the invention for the amounts of the compound of the present invention (referred to as “the compound”) and the one or more additional therapeutic agent(s) to be subtherapeutic by themselves, but the combination of the compound of the present invention and the one or more additional therapeutic agent(s) is therapeutic.

Co-administration of the compound of the present invention with one or more other active agents generally refers to simultaneous or sequential administration of the compound and one or more other active agents, such that the compound and one or more other active agents are both present in the body of the patient. Simultaneous administration of the compound and one or more additional therapeutic agents can be achieved, for example, by mixing the compound and one or more additional therapeutic agents in a single dosage form, such as a tablet or injectable solution. Again by way of example, simultaneous administration of the compound and one or more additional therapeutic agents can be achieved by co-packaging, for example in a blister pack, the compound and at least one other therapeutic agent, so that a patient can remove and consume individual doses of the compound and the other therapeutic agent.

Co-administration includes administration of unit dosages of the compound before or after administration of unit dosages of one or more other active agents, for example, administration of the compound within seconds, minutes, or hours of the administration of one or more other active agents. For example, a unit dose of the compound can be administered first, followed within seconds or minutes by administration of a unit dose of one or more other active agents. Alternatively, a unit dose of one or more other active agents can be administered first, followed by administration of a unit dose of the compound within seconds or minutes. In some cases, it may be desirable to administer a unit dose of the compound first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of the compound.

In still yet another embodiment, the present application provides for the use of a compound of the present invention, or a pharmaceutically acceptable salt thereof, for the preparation of a medicament for treating an HCV infection.

In general it is contemplated that an antiviral effective daily amount would be from 0.01 mg/kg to 500 mg/kg body weight, more preferably from 0.1 mg/kg to 50 mg/kg body weight. It may be appropriate to administer the required dose as two, three, four or more sub-doses at appropriate intervals throughout the day. Said sub-doses may be formulated as unit dosage forms, for example, containing 1 to 1000 mg, and in particular 5 to 200 mg of active ingredient per unit dosage form.

The exact dosage and frequency of administration depends on the particular compound of formula (I) or (II) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention. The effective daily amount ranges mentioned hereinabove are therefore only guidelines.

In one embodiment of the present invention there is provided an article of manufacture comprising a composition effective to treat an HCV infection or to inhibit the NS3 protease of HCV; and packaging material comprising a label which indicates that the composition can be used to treat infection by the hepatitis C virus; wherein the composition comprises a compound of the formula (I) or (II) or any subgroup thereof, or the combination as described herein.

### EXAMPLES

The following examples are intended to illustrate the present invention and not to limit it thereto.

### Preparation of Sulfamic Acid 1-methylcyclopropyl Ester

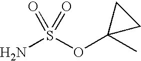

1-Methyl-cyclopropanol was synthesized according to a previously published procedure (Synthesis 1991, 3, 234). Alterations to the workup procedure were employed to improve yield and minimize unwanted byproducts. After acidic quench of the reaction, the separated organic layer was stirred vigorously over basic alumina and PDC on silica (20% loading) for 10 mins. MgSO₄ was then added to further dry the organics and the mixture was filtered through a silica gel plug. After removal of solvents, the residual slightly yellow liquid was used directly in the following esterification without further purification.

A three-necked round bottom equipped with a reflux condenser was charged with chlorosulfonyl isocyanate (5.25 ml, 0.06 mol) and cooled to 0° C. Formic acid (2.25 mL, 0.06 mol) was added dropwise with rapid stirring and rapid gas evolution was observed. Upon complete addition of formic acid, the reaction was allowed to warm to room temperature. After 2 h, the resultant reaction vessel containing the solid sulfamoyl chloride was cooled to 0° C. and 1-methylcyclopropanol (2g, ±0.02 mol) dissolved in NMP (25 mL) was added dropwise via an addition funnel. The reaction was allowed to warm to room temperature. After 3 h stirring, the reaction mixture was poured into cold saturated aqueous NaCl (120 mL) and extracted with EtOAc. After removal of the separated organic solvent, the crude product was purified by column chromatography on silica (35% EtOAc/hexane) to provide sulfamic acid 1-methylcyclopropyl ester (1.6 g, 53%): ¹H-NMR (CDCl₃, 300 MHz) δ 4.83 (bs, 2H), 1.70 (s, 3H), 1.32 (m, 2H), 0.68 (m, 2H).

### Preparation of (1R,2S)-1-amino-2-vinylcyclopropanecarbonyl)-sulfamic acid 1-methylcyclopropyl ester hydrochloride

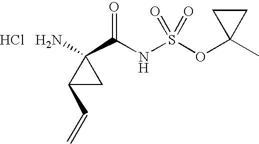

### Step 1: (1R,2S)-[2-Vinyl-1-(1-methylcyclopropoxysulfonylaminocarbonypcyclopropyl]-carbamic acid tert-butyl ester

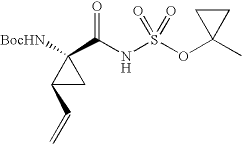

To a solution of (1R,2S)-1-tert-butoxycarbonylamino-2-vinyl-cyclopropanecarboxylic acid (Wang, et al. WO2003/099274; 1.00 g, 4.40 mmol) in CH₂Cl₂ (22 mL) was added sulfamic acid 1-methylcyclopropyl ester (0.328 g, 2.20 mmol), HATU (1.85 g, 4.84 mmol) and DIPEA (3.8 mL, 22 mmol). The reaction mixture was stirred at room temperature for 3 days before dilution with CH₂Cl₂. The solution was washed twice with aqueous HCl (1M) and once with brine. The aqueous layers were backextracted with CH₂Cl₂. The organic layers were combined, dried over Na₂SO₄, and concentrated in vacuo. The crude sulfamate was purified by column chromatography (20→100% EtOAc/hexanes) to provide (1R,2S)-[2-vinyl-1-(1-methyl-cyclopropoxysulfonylamino-carbonyl)-cyclopropyl]-carbamic acid tert-butyl ester (1.39 g, 88%). (LC/MS: m/z 382.9 (M+Na)⁺). ¹H-NMR (CDCl₃, 400 MHz) δ 5.64-5.52 (m, 1H), 5.32-5.28 (m, 2H), 5.17 (dd, 1H), 2.14 (q, 1H), 1.89 (dd, 1H), 1.69 (s, 3H), 1.48 (s, 9H), 1.34-1.28 (m, 3H), 0.68-0.64 (m, 2H).

### Step 2: (1R,2S)-1-Amino-2-vinylcyclopropanecarbonyl)-sulfamic acid 1-methyl-cyclopropyl ester hydrochloride

To a solution of (1R,2S)-[2-vinyl-1-(1-methyl-cyclopropoxysulfonylamino-carbonyl)-cyclopropyl]-carbamic acid tert-butyl ester (0.799 g, 2.21 mmol) in CH₂Cl₂ (4.5 mL) was slowly added 4M HCl in dioxane (11 mL, 44 mmol). After 3 h, the volatiles are removed in vacuo to afford a quantitive yield of (1R,2R)-1-amino-2-ethylcyclopropanecarbonyl)-sulfamic acid 1-methyl-cyclopropyl ester hydrochloride, which was used in future couplings without further purification.

### BIOLOGICAL ASSAYS

NS3 Enzymatic Potency: Purified NS3 protease is complexed with NS4A peptide and then incubated with serial dilutions of compound (DMSO used as solvent). Reactions are started by addition of dual-labeled peptide substrate and the resulting kinetic increase in fluorescence is measured. Non-linear regression of velocity data is performed to calculate IC₅₀s. Activity is initially tested against genotype 1b protease. Depending on the potency obtained against genotype 1b, additional genotypes (1a, 2a, 3) and or protease inhibitor resistant enzymes (D168Y, D168V, or A156T mutants) may be tested. BILN-2061 is used as a control during all assays. Representative compounds of the invention were evaluated in this assay and were typically found to have IC₅₀ values of less than about 1 μm.

Replicon Potency and Cytotoxicity: Huh-luc cells (stably replicating Bartenschlager's I3891uc-ubi-neo/NS3-3′/ET genotype 1b replicon) is treated with serial dilutions of compound (DMSO is used as solvent) for 72 hours. Replicon copy number is measured by bioluminescence and nonlinear regression is performed to calculate EC₅₀s. Parallel plates treated with the same drug dilutions are assayed for cytotoxicity using the Promega CellTiter-Glo cell viability assay. Depending on the potency achieved against the 1b replicon, compounds may be tested against a genotype 1a replicon and/or inhibitor resistant replicons encoding D168Y or A156T mutations. BILN-2061 is used as a control during all assays. Representative compounds of the invention were evaluated in this assay and were typically found to have EC₅₀ values of less than about 5 μm.

### Effect of Serum Proteins on Replicon Potency

Replicon assays are conducted in normal cell culture medium (DMEM+10% FBS) supplemented with physiologic concentrations of human serum albumin (40 mg/mL) or a-acid glycoprotein (1 mg/mL). EC₅₀s in the presence of human serum proteins are compared to the EC₅₀ in normal medium to determine the fold shift in potency.

Enzymatic Selectivity: The inhibition of mammalian proteases including Porcine Pancreatic Elastase, Human Leukocyte Elastase, Protease 3, and Cathepsin D are measured at Kₘ for the respective substrates for each enzyme. IC₅₀ for each enzyme is compared to the IC₅₀ obtained with NS31b protease to calculate selectivity. Representative compounds of the invention have shown activity.

MT-4 Cell Cytotoxicity: MT4 cells are treated with serial dilutions of compounds for a five day period. Cell viability is measured at the end of the treatment period using the Promega CellTiter-Glo assay and non-linear regression is performed to calculate CC₅₀.

Compound Concentration Associated with Cells at EC₅₀: Huh-luc cultures are incubated with compound at concentrations equal to EC₅₀. At multiple time points (0-72 hours), cells are washed 2× with cold medium and extracted with 85% acetonitrile; a sample of the media at each time-point will also be extracted. Cell and media extracts are analyzed by LC/MS/MS to determine the Molar concentration of compounds in each fraction. Representative compounds of the invention have shown activity.

Solubility and Stability: Solubility is determined by taking an aliquot of 10 mM DMSO stock solution and preparing the compound at a final concentration of 100 μM in the test media solutions (PBS, pH 7.4 and 0.1 N HCl, pH 1.5) with a total DMSO concentration of 1%. The test media solutions are incubated at room temperature with shaking for 1 hr. The solutions will then be centrifuged and the recovered supernatants are assayed on the HPLC/UV. Solubility will be calculated by comparing the amount of compound detected in the defined test solution compared to the amount detected in DMSO at the same concentration. Stability of compounds after an 1 hour incubation with PBS at 37° C. will also be determined.

Stability in Cryopreserved Human, Dog, and Rat Hepatocytes: Each compound is incubated for up to 1 hour in hepatocyte suspensions (100 μl 80,000 cells per well) at 37° C. Cryopreserved hepatocytes are reconstituted in the serum-free incubation medium. The suspension is transferred into 96-well plates (50 μL/well). The compounds are diluted to 2 μM in incubation medium and then are added to hepatocyte suspensions to start the incubation. Samples are taken at 0, 10, 30 and 60 minutes after the start of incubation and reaction will be quenched with a mixture consisting of 0.3% formic acid in 90% acetonitrile/10% water. The concentration of the compound in each sample is analyzed using LC/MS/MS. The disappearance half-life of the compound in hepatocyte suspension is determined by fitting the concentration-time data with a monophasic exponential equation. The data will also be scaled up to represent intrinsic hepatic clearance and/or total hepatic clearance.

Stability in Hepatic S9 Fraction from Human, Dog, and Rat: Each compound is incubated for up to 1 hour in S9 suspension (500 μl, 3 mg protein/mL) at 37° C. (n=3). The compounds are added to the S9 suspension to start the incubation. Samples are taken at 0, 10, 30, and 60 minutes after the start of incubation. The concentration of the compound in each sample is analyzed using LC/MS/MS. The disappearance half-life of the compound in 59 suspension is determined by fitting the concentration-time data with a monophasic exponential equation.

Caco-2 Permeability: Compounds are assayed via a contract service (Absorption Systems, Exton, Pa.). Compounds are provided to the contractor in a blinded manner. Both forward (Ato-B) and reverse (B-to-A) permeability will be measured. Caco-2 monolayers are grown to confluence on collagen-coated, microporous, polycarbonate membranes in 12-well Costar Transwell® plates. The compounds are dosed on the apical side for forward permeability (A-toB), and are dosed on the basolateral side for reverse permeability (B-to-A). The cells are incubated at 37° C. with 5% CO₂ in a humidified incubator. At the beginning of incubation and at 1 hr and 2 hr after incubation, a 200-μL, aliquot is taken from the receiver chamber and replaced with fresh assay buffer. The concentration of the compound in each sample is determined with LC/MS/MS. The apparent permeability, Papp, is calculated.

### Plasma Protein Binding:

Plasma protein binding is measured by equilibrium dialysis. Each compound is spiked into blank plasma at a final concentration of 2 μM. The spiked plasma and phosphate buffer is placed into opposite sides of the assembled dialysis cells, which will then be rotated slowly in a 37° C. water bath. At the end of the incubation, the concentration of the compound in plasma and phosphate buffer is determined. The percent unbound is calculated using the following equation:

$$\% Unbound = 100 \cdot ( \frac{{C}_{f}}{{C}_{b} + {C}_{f}} )$$

Where C_{f} and C_{b} are free and bound concentrations determined as the post-dialysis buffer and plasma concentrations, respectively.

### CYP450 Profiling:

Each compound is incubated with each of 5 recombinant human CYP450 enzymes, including CYP1A2, CYP2C9, CYP3A4, CYP2D6 and CYP2C19 in the presence and absence of NADPH. Serial samples will be taken from the incubation mixture at the beginning of the incubation and at 5, 15, 30, 45 and 60 min after the start of the incubation. The concentration of the compound in the incubation mixture is determined by LC/MS/MS. The percentage of the compound remaining after incubation at each time point is calculated by comparing with the sampling at the start of incubation.

### Stability in Rat, Don, Monkey and Human Plasma:

Compounds will be incubated for up to 2 hours in plasma (rat, dog, monkey, or human) at 37° C. Compounds are added to the plasma at final concentrations of 1 and 10 μg/mL. Aliquots are taken at 0, 5, 15, 30, 60, and 120 min after adding the compound. Concentration of compounds and major metabolites at each timepoint are measured by LC/MS/MS.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound having the formula 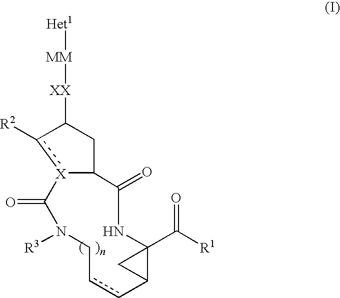
R¹ is: 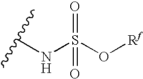
an N-oxide, salt, or stereoisomer thereof, wherein each dashed line (represented by ----) represents an optional double bond;
X is N, CH and where X bears a double bond it is C;
R¹ is —NH—SO₂(OR^{f});
R² is hydrogen, and where X is C or CH, R² may also be C₁₋₆alkyl;
R³ is hydrogen, C₁₋₆alkyl, C₃₋₇cycloalkyl;
R⁴ is aryl or Het;
n is 3, 4, 5, or 6;
carbon atoms bearing four substituents and including at least one bond to hydrogen in a compound of structure (I) may optionally have one or more of their hydrogen atoms replaced by halo where the halo can be F, Cl, Br or I, preferably F;
R⁵ represents halo, C₁₋₆alkyl, hydroxy, alkoxy, polyhaloC₁₋₆ alkyl, phenyl, or Het;
R⁶ represents C₁₋₆ alkoxy, dimethylamino or mono- or di-C₁-6alkylamino; aryl as a group or part of a group is phenyl or naphthyl optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, 6alkylcarbonyl, amino, mono- or di-C₁₋₆alkylamino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkylpiperazizyl, 4-C₁₋₆alkylcarbonylpiperazinyl, and morpholinyl; wherein the morpholinyl and piperidinyl groups may be optionally substituted with one or with two 6alkyl radicals;
Het as a group or part of a group is a 5 or 6 membered saturated, partially unsaturated or completely unsaturated heterocyclic ring containing 1 to 4 heteroatoms each independently selected from nitrogen, oxygen and sulfur, said heterocyclic ring being optionally condensed with a benzene ring; and wherein said Het as a whole is optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or di-C₁₋₆alkylamino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkylcarbonylpiperazinyl, and morpholinyl; wherein the morpholinyl and piperidinyl groups may be optionally substituted with one or with two C₁₋₆alkyl radicals;
R^{f} is A³;
Het¹ is a heterocycle or aryl group and can optionally be substituted with up to two Het and up to five groups selected independently from R⁴, R⁵ or R⁶;
MM is CO or a bond;
XX is O, NH, N(C₁₋₄alkyl), a bond, or CH₂;
A³ is independently selected from PRT, H, —OH, —C(O)OH, cyano, alkyl, alkenyl, alkynyl, amino, amido, imido, imino, halogen, CF₃, CH₂CF₃, cycloalkyl, nitro, aryl, aralkyl, alkoxy, aryloxy, heterocycle, —C(A²)₃, —C(A²)₂—C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(A²)(OA²), —OCH₂P(Y¹)(A²)(OA²), —OCH₂P(Y¹)(A²)(N(A²)₂), —C(O)OCH₂P(Y¹)(OA²)(OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(r)(OA²)(N (A²)₂), —CH₂P(Y¹)(N(A²)₂)(N(A²)₂), —C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O —(CH₂)ₘ—O—C(O)—Oalkyl, —O—(CH₂)ₘ—O—C(O)—(CH₂)ₘ-alkyl, —(CH₂)ₘO—C(O)—O-alkyl, —(CH₂)ₘO—C(O)—O-cycloalkyl, —N(H)C(Me)C(O)O-alkyl, SRᵣS(O)Rᵣ, S(O)₂Rᵣ, or alkoxy arylsulfamate,
wherein each A³ may be optionally substituted with 1 to 4
—R¹¹¹, —P(Y¹)(OA²)(OA²), —P(Y¹)(OA²)(N(A²)₂), —P(Y¹)(A²)(OA²), P(Y¹)(A²)(N(A²)₂), or P(Y¹)(N(A²)₂)(N(A²)₂), —C(—O)N(A²)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘ—O—C(O)—O-alkyl, —O—(CH₂)ₘ—O—C(O)—(CH₂)ₘ-alkyl, —(CH₂)ₘ—O—C(O)—O-alkyl, —(CH₂)ₘ—O—C(O)-β-cycloalkyl, —N(H)C(CH₃)C(O)O-alkyl, or alkoxy arylsulfonamide, optionally substituted with R¹¹¹;
A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;
R¹¹¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;
Y¹ is independently O, S, N(A³), N(O)(A³), N(OA³), N(O)(OA³) or N(N(A³)(A³));
m is 0 to 6;
r is 0 to 6.

2. A compound of claim 1 with the structure (II): 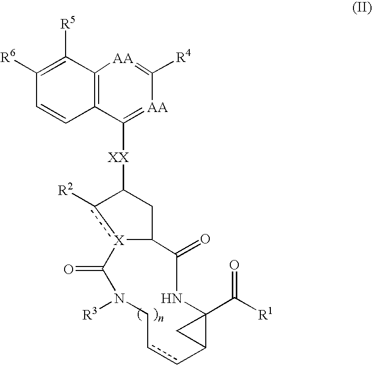
wherein AA is independently N or CH.

3. A compound according to claim 1, wherein the compound has the formula (I-c), (I-d), or (I-e):

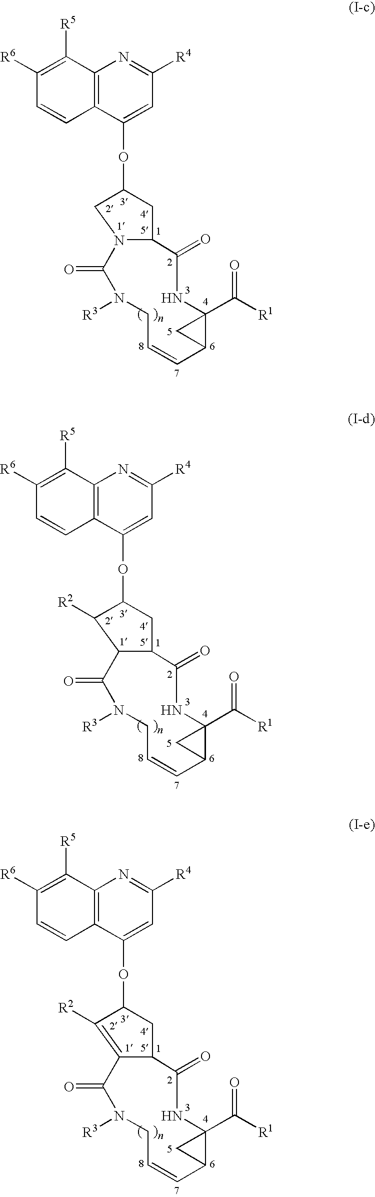

4. A compound according to claim 1, wherein R⁴ is selected from the group consisting of phenyl, pyridin-4-yl, 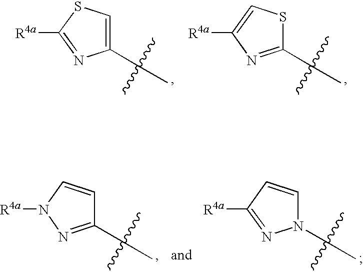
wherein R^{4a} is, each independently, hydrogen, halo, C₁₋₆alkyl, amino, or mono- or di-C₁₋₆alkylamino.

5. A compound according to claim 2, wherein R⁵ is methyl, ethyl, isopropyl, tent-butyl, fluoro, chloro, or bromo; and R⁶ is methoxy.

6. A compound according to claim 2, wherein n is 4 or 5.

7. A compound according to claim 1, wherein R³ is hydrogen or C₁₋₆alkyl, in particular R³ is hydrogen or methyl.

8. A compound according to claim 1, wherein R⁴ is a radical 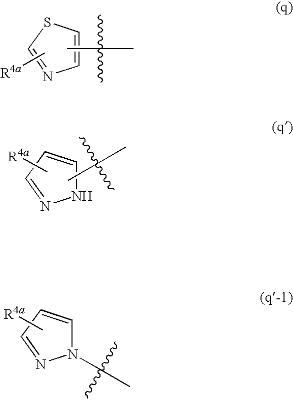
wherein, where possible a nitrogen may bear an R^{4a} substituent or a link to the remainder of the molecule; each R^{4a} in any of the R⁴ substituents may be selected from those mentioned as possible substituents on Het, as specified in claim 1.

9. A compound according to claim 1, wherein R⁴ is selected from the group consisting of 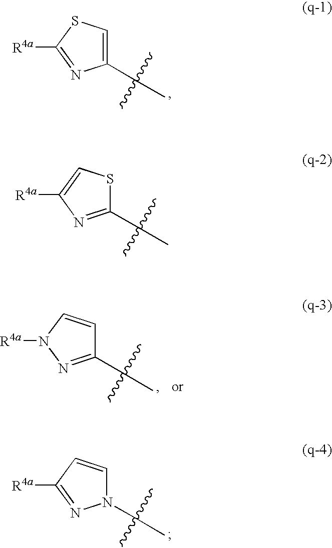
wherein each R^{4a} is hydrogen, halo, C₁₋₆alkyl, amino, or mono- or di-C₁₋₆alkylamino, pyrrolidinyl, piperidinyl, morpholinyl, piperazinyl, 4-C₁₋₆alkylpiperazinyl; and wherein the morpholinyl and piperidinyl groups may optionally substituted with one or two C₁₋₆alkyl radicals;

10. A compound according to claim 1, wherein R⁶ is methoxy.

11. A compound according to claim 1 wherein the compound is: 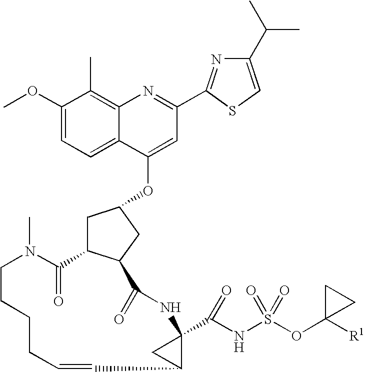
wherein R^{f} is A³.

12. A compound according to claim 1 wherein the compound is: 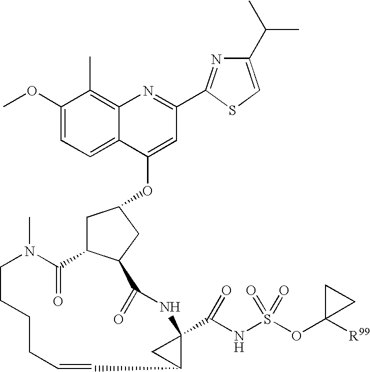
wherein R⁹⁹ is H, methyl, C₂₋₈alkyl, C₂₋₈haloalkyl or C₁₋₆alkoxy.

13. A compound according to claim 1 wherein the compound is: 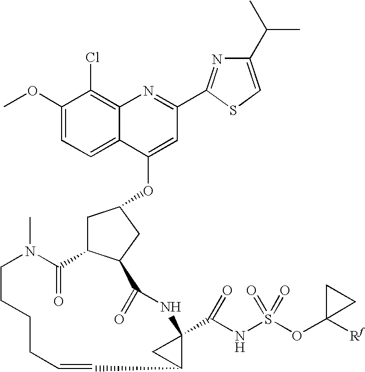
wherein R¹ is A³.

14. A compound according to claim 1 wherein the compound is: 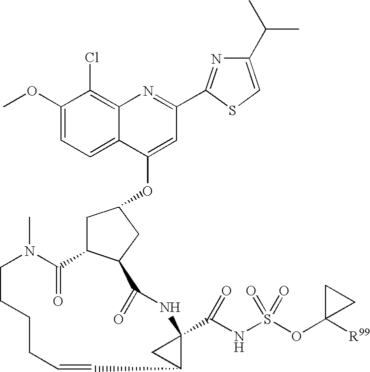
wherein R⁹⁹ is H, methyl, C₂₋₈ alkyl, C₂₋₈ haloalkyl or C₁₋₆ alkoxy.

15. A compound according to claim 1 other than an N-oxide, or salt.

16. A pharmaceutical composition comprising a carrier, and as active ingredient an anti-virally effective amount of a compound as claimed in claim 1 or a combination of compounds of claim 1.

17. A method of inhibiting HCV replication in a warm-blooded animal said method comprising the administration of an effective amount of a compound according to claim 1.

18. A process for preparing a compound as claimed in claim 1, wherein said process comprises:
(a) preparing a compound of formula (I) wherein the bond between C7 and C8 is a double bond, which is a compound of formula (I-i), by forming a double bond between C7 and C8, in particular via an olefin metathesis reaction, with concomitant cyclization to the macrocycle as outlined in the following reaction scheme: 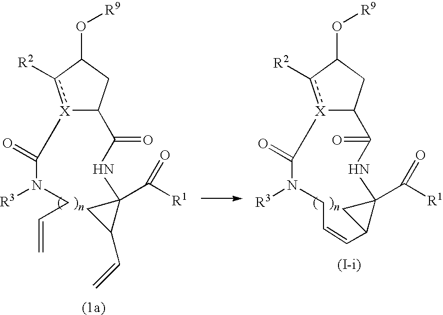
wherein in the above and following reaction schemes R⁹ represents Het¹ or a radical 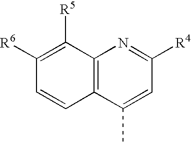
(b) converting a compound of formula (I-i) to a compound of formula (I) wherein the link between C7 and C8 in the macrocycle is a single bond, i.e. a compound of formula (I-j): 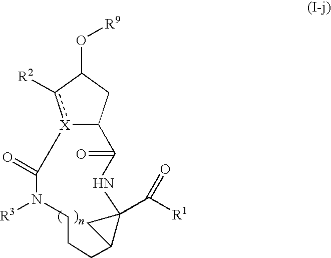
by a reduction of the C7-C8 double bond in the compounds of formula (I-j);
(c) preparing a compound of formula (I), said compounds being represented by formula (I-k-1), by forming an amide bond between an intermediate (2a) and a sulfamate (2b), as outlined in the following scheme wherein G represents a group: 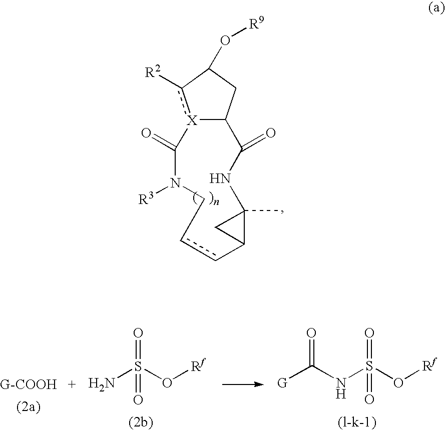
(d) preparing a compound of formula (I) wherein R³ is hydrogen, said compound being represented by (I-1), from a corresponding nitrogen-protected intermediate (3a), wherein PG represents a nitrogen protecting group: 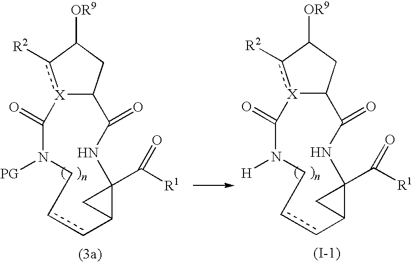
(e) reacting an intermediate (4a) with intermediate (4b) as outlined in the following reaction scheme: 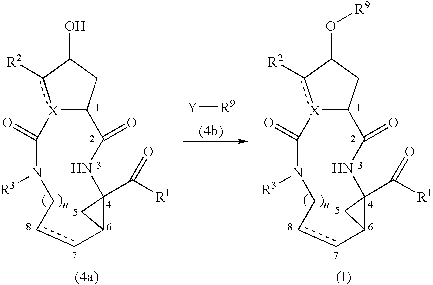
wherein Y in (4b) represents hydroxy or a leaving group; and where Y represents hydroxy the reaction of (4a) with (4b) is a Mitsunobu reaction; and where Y represents a leaving group the reaction of (4a) with (4b) is a substitution reaction;
(f) converting compounds of formula (I) into each other by a functional group transformation reaction; or
(g) preparing a salt form by reacting the free form of a compound of formula (I) with an acid or a base.

19. The compound of claim 1 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;
each Rₕ and Rᵢ is independently H, alkyl, or haloalkyl; and
R_{d} is H, (C₁-C₁₀)alkyl, or aryl, which is optionally substituted with one or more halo.

20. The compound of claim 2 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
wherein each alkyl of R_{g} is optionally substituted with one or more halo, alkoxy, or cyano;
each Rₕ and R, is independently H, alkyl, or haloalkyl; and
R_{d} is H, (C₁-C₁₀)alkyl, or aryl, which is optionally substituted with one or more halo.

21. The compound of claim 3 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;
each Rₕ and Rᵢ is independently H, alkyl, or haloalkyl; and
R_{d} is H, (C₁-10)alkyl, or aryl, which is optionally substituted with one or more halo.

22. The compound of claim 1 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and Rᵢ is independently H, alkyl, or haloalkyl.

23. The compound of claim 2 wherein R^{f} is H, alkyl, alkenyl, alkynyl, aryl, heteroaryl, or cycloalkyl, which 1e is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ—C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and Rᵢ is independently H, alkyl, or haloalkyl.

24. The compound of claim 3 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ,
wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and Rᵢ, is independently H, alkyl, or haloalkyl.

25. The compound of claim 1 wherein R^{f} is alkyl, aryl, cycloalkyl, which R^{f} is optionally substituted with one or more R^{g} independently selected from alkyl, halo, —C(═O)OR_{d}, or trifluoromethyl, wherein each alkyl of R_{g} is optionally substituted with one or more halo, alkoxy, or cyano.

26. The compound of claim 2 wherein R^{f} is alkyl, aryl, cycloalkyl, which R^{f} is optionally substituted with one or more R_{g} independently selected from alkyl, halo, —C(═O)OR_{d}, or trifluoromethyl, wherein each alkyl of R_{g} is optionally substituted with one or more halo, alkoxy, or cyano.

27. The compound of claim 3 wherein R^{f} is alkyl, aryl, cycloalkyl, which R^{f} is optionally substituted with one or more R_{g} independently selected from alkyl, halo, —C(═O)OR_{d}, or trifluoromethyl, wherein each alkyl of R^{g} is optionally substituted with one or more halo, alkoxy, or cyano.

28. The compound of claim 1 wherein R^{f} is aryl, heteroaryl, or cycloalkyl, which R^{f} is optionally substituted with one to three A³.

29. The compound of claim 2 wherein R^{f} is aryl, heteroaryl, or cycloalkyl, which R^{f} is optionally substituted with one to three A³.

30. The compound of claim 3 wherein R^{f} is aryl, heteroaryl, or cycloalkyl, which R^{f} is optionally substituted with one to three A³.

31. The compound of claim 1 wherein R^{f} is cyclopropyl which R^{f} is optionally substituted by up to four A³.

32. The compound of claim 2 wherein R^{f} is cyclopropyl which R^{f} is optionally substituted by up to four A³.

33. The compound of claim 3 wherein R^{f} is cyclopropyl which R^{f} is optionally substituted by up to four A³.

34. The compound of claim 1 wherein R^{f} is cyclopropyl which R^{f} is optionally substituted by up to three C₁₋₆ alkyl.

35. The compound of claim 2 wherein R^{f} is cyclopropyl which R^{f} is optionally substituted by up to three C₁₋₅ alkyl.

36. The compound of claim 3 wherein R^{f} is cyclopropyl which R^{f} is optionally substituted by up to three C₁₋₆ alkyl.

37. The compound of claim 1 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

38. The compound of claim 2 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

39. The compound of claim 3 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

40. The compound of claim 1 wherein R^{f} is cyclopropyl.

41. The compound of claim 2 wherein R^{f} is cyclopropyl.

42. The compound of claim 3 wherein R^{f} is cyclopropyl.

43. The compound of claim 1 wherein R^{f} is 1-methylcyclopropyl.

44. The compound of claim 2 wherein R^{f} is 1-methylcyclopropyl.

45. The compound of claim 3 wherein R^{f} is 1-methylcyclopropyl.

46. The compound of claim 1 wherein the compound is:

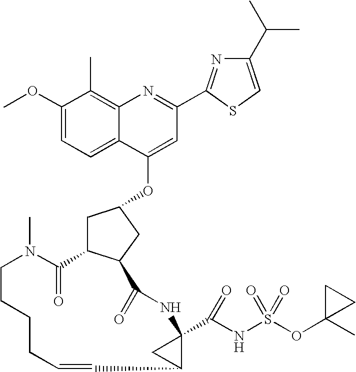

47. The compound of claim 1 wherein the compound is:

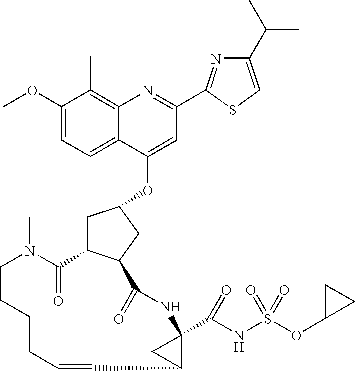

48. The compound of claim 1 wherein the compound is:

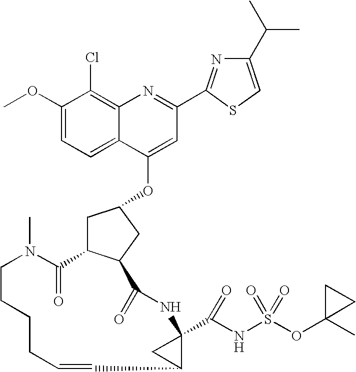

49. The compound of claim 1 wherein the compound is: [chemical structure]
